(19) United States
(12) Patent Application Publication
(10) Pub. No.: US 2009/0299421 A1
(43) Pub. Date: Dec. 3, 2009

(54) EVALUATION OF IMPLANTABLE MEDICAL DEVICE SENSING INTEGRITY BASED ON EVOKED SIGNALS

(75) Inventor: Robert T. Sawchuk, Roseville, MN (US)

Correspondence Address: Medtronic, Inc., 710 Medtronic Parkway, Minneapolis, MN, 55432, US

(73) Assignee: Medtronic, Inc., Minneapolis (US)

(21) Appl. No.: 12/179,166

(22) Filed: Jul. 24, 2008

Related U.S. Application Data
(60) Provisional application No. 61/058,105, filed on Jun. 2, 2008.

(51) Int. Cl. A61N 1/39; A61N 1/37

(52) U.S. Cl. 607/4; 607/5; 607/28

(57) ABSTRACT

The disclosure describes techniques for evaluating sensing integrity of an implantable medical device (IMD) based on sensing of evoked signals. Sensing integrity may provide an indication of reliability of implantable leads associated with an IMD. The sensed signals may be signals that are evoked by tissue in response to delivery of electrical stimulation. The techniques may involve evaluation of sensing integrity based on sensing of evoked cardiac potentials generated in response to cardiac stimulation, such as pacing pulses. Signals evoked in response to electrical stimulation may be measured and trended to permit analysis of evoked signals over time. Lead integrity may be inferred from sensing integrity. By analyzing evoked signals, sensing integrity may be evaluated without sensing intrinsic events. Evaluation of sensing integrity can facilitate analysis in the presence of pacing, including pacing delivered by IMDs that pace substantially continuously, such as IMDs configured to support cardiac resynchronization therapy (CRT).

This application claims the benefit of U.S. Provisional Application No. 61/058,105, filed Jun. 2, 2008, the entire content of which is incorporated herein by reference.

### TECHNICAL FIELD

The disclosure relates to implantable medical devices and, more particularly, to evaluating sensing integrity of an implantable medical device.

### BACKGROUND

A variety of implantable medical devices for delivering a therapy and/or monitoring a physiological condition have been clinically implanted or proposed for clinical implantation in patients. Some implantable medical devices may employ one or more elongated electrical leads carrying stimulation electrodes, sense electrodes, and/or other sensors. Implantable medical devices may deliver electrical stimulation or fluid therapy and/or monitor conditions associated with the heart, muscle, nerve, brain, stomach or other organs or tissue. Implantable medical leads may be configured to allow electrodes or other sensors to be positioned at desired locations for delivery of stimulation or sensing. For example, electrodes or sensors may be carried at a distal portion of a lead. A proximal portion of the lead may be coupled to an implantable medical device housing, which may contain circuitry such as stimulation generation and/or sensing circuitry.

Implantable medical devices, such as cardiac pacemakers or implantable cardioverter-defibrillators, for example, provide therapeutic electrical stimulation to the heart via electrodes carried by one or more implantable leads. The electrical stimulation may include signals such as pulses or shocks for pacing, cardioversion or defibrillation. In some cases, an implantable medical device may sense intrinsic depolarizations of the heart, and control delivery of stimulation signals to the heart based on the sensed depolarizations. Upon detection of an abnormal rhythm, such as bradycardia, tachycardia or fibrillation, an appropriate electrical stimulation signal or signals may be delivered to restore or maintain a more normal rhythm. For example, in some cases, an implantable medical device may deliver pacing pulses to the heart of the patient upon detecting tachycardia or bradycardia, and deliver cardioversion or defibrillation shocks to the heart upon detecting fibrillation.

Leads associated with an implantable medical device typically include a lead body containing one or more elongated electrical conductors that extend through the lead body from a connector assembly provided at a proximal lead end to one or more electrodes located at the distal lead end or elsewhere along the length of the lead body. The conductors connect stimulation and/or sensing circuitry within an associated implantable medical device housing to respective electrodes or sensors. Some electrodes may be used for both stimulation and sensing. Each electrical conductor is typically electrically isolated from other electrical conductors and is encased within an outer sheath that electrically insulates the lead conductors from body tissue and fluids.

Cardiac lead bodies tend to be continuously flexed by the beating of the heart. Other stresses may be applied to the lead body during implantation or lead repositioning. Patient movement can cause the route traversed by the lead body to be constricted or otherwise altered, causing stresses on the lead body. The electrical connection between implantable medical device connector elements and the lead connector elements can be intermittently or continuously disrupted. Connection mechanisms, such as set screws, may be insufficiently tightened at the time of implantation, followed by a gradual loosening of the connection. Also, lead pins may not be completely inserted. In some cases, changes in leads or connections may result in intermittent or continuous changes in lead impedance.

Short circuits, open circuits or significant changes in impedance may be referred to, in general, as lead related conditions. In the case of cardiac leads, sensing of an intrinsic heart rhythm through a lead can be altered by lead related conditions. Structural modifications to leads, conductors or electrodes may alter sensing integrity. Furthermore, impedance changes in the stimulation path due to lead related conditions may affect sensing and stimulation integrity for pacing, cardioversion, or defibrillation. In addition to lead related conditions, conditions associated with sensor devices or sensing circuitry may affect sensing integrity.

### SUMMARY

In general, the disclosure describes techniques for evaluating sensing integrity of an implantable medical device (IMD) based on sensing of evoked signals. Sensing integrity may provide an indication of the reliability of one or more implantable leads, stimulation electrodes, sense electrodes, other sensors, and/or sensing circuitry associated with an IMD. As an example, the sensed signals may be signals that are evoked by stimulated tissue in response to delivery of electrical stimulation. In this case, the techniques may involve evaluation of sensing integrity based on sensing of evoked cardiac potentials generated in response to cardiac stimulation, such as pacing pulses.

Signals that are evoked in response to electrical stimulation may be measured and processed to permit analysis of sensed evoked signals over time. In some cases, lead integrity and lead-related conditions may be inferred from sensing integrity. By analyzing the sensing of evoked signals, sensing integrity may be evaluated without the need to sense intrinsic events. Evaluation of sensing integrity in this manner can facilitate analysis in the presence of pacing. The disclosed techniques may be especially useful in IMDs that pace substantially continuously, such as IMDs configured to support cardiac resynchronization therapy (CRT).

In one example, the disclosure is directed to a method comprising obtaining sensed signals evoked by tissue in response to electrical stimulation of the tissue by an implantable medical device, and evaluating sensing integrity of the implantable medical device based on analysis of the sensed signals.

In another example, the disclosure is directed to an implantable medical device system comprising an implantable stimulation generator configured to deliver electrical stimulation to tissue, an implantable sensing module configured to sense signals evoked by the tissue in response to the electrical stimulation of the tissue by the implantable stimulation generator, implantable memory configured to store data relating to the sensed signals, and an evaluation unit configured to support evaluation of sensing integrity of the implantable sensing module based on analysis of the stored data.

The techniques described in this disclosure may be implemented in hardware, software, firmware, or a combination thereof. If implemented in software, the software may be executed by one or more processors. The software may be initially stored in a computer readable storage medium and loaded by a processor for execution. Accordingly, this disclosure contemplates computer-readable media comprising instructions to cause one or more processors to perform techniques as described in this disclosure.

The details of one or more aspects of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example therapy system that may be used to provide cardiac stimulation therapy to a patient.

FIG. 2A is a conceptual diagram illustrating a portion of the example therapy system of FIG. 1 in greater detail.

FIG. 2B is conceptual diagram illustrating a portion of another example therapy system similar to the system of FIG. 2A.

FIG. 3 is a conceptual diagram illustrating another example of a cardiac therapy system.

FIG. 4A is a functional block diagram illustrating various components of an example implantable medical device.

FIG. 4B is a functional block diagram illustrating various components of another example implantable medical device similar to the device of FIG. 4A.

FIG. 5 is a block diagram illustrating various components of an example programmer for programming an implantable medical device.

FIG. 6 is a flow chart illustrating an example technique for evaluating sensing integrity based on sensing of evoked signals according to an aspect of the disclosure.

FIG. 7 is a schematic diagram illustrating use of various sensing vectors to sense evoked signals.

FIG. 8 is a flow chart illustrating an example technique for evaluating sensing integrity in more detail.

FIG. 9A is a graphical representation of an example of a single sensed evoked cardiac signal waveform.

FIG. 9B is a graphical representation of an example series of sensed evoked cardiac signal waveforms.

FIGS. 10A and 10B are graphical representations of trend data produced for sensed evoked cardiac signal waveforms over time.

FIG. 11 is a flow diagram illustrating an example technique for evaluating sensing integrity using various statistical measures.

FIG. 12 is a block diagram illustrating an example system that includes an external device, such as a server, and one or more computing devices that are coupled to the IMD and programmer shown in FIG. 1 via a network.

### DETAILED DESCRIPTION

In general, the disclosure describes techniques for evaluating sensing integrity of an implantable medical device (IMD) based on sensing of evoked signals. Sensing integrity may be a function of one or more factors, such as the reliability of one or more implantable leads, including associated electrical conductors, contacts and electrodes, the reliability of sensors such as sense electrodes or other types of sensors, and the reliability of electronic sensing circuitry within the IMD or coupled to the IMD via the electrical conductors.

Sensing integrity, in general, may refer to the ability of the IMD to accurately and reliably sense particular events in order to properly record such events and/or control therapy in response to such events. Evaluation of sensing integrity may also provide an indication of lead integrity, particularly to the extent sensing makes use of contacts, conductors and electrodes associated with an implantable lead. Reliability of contacts, conductors and electrodes may impact the ability of the IMD to not only accurately sense particular events, but also reliably deliver electrical stimulation therapy via the leads. Further, the ability of other types of sensors, such as lead-based electronic sensors, to convey sensed information, e.g., via a lead to the IMD and/or associated programmer, may be impacted by lead integrity.

Various techniques, as described in this disclosure, may be applied to sense signals evoked by tissue in response to delivery of electrical stimulation to the tissue, analyze the sensed signals, and evaluate sensing integrity based on the analysis. Various characteristics of a sensed evoked signal, including amplitude, frequency, signal morphology, or other characteristics, may provide an indication of sensing integrity. Examples of such characteristics include but are not limited to signal amplitude and frequency, the relative area under a curve created by a sensed signal waveform, the slope of a curve created by a sensed signal, and inflection points of a curve created by a sensed signal. In turn, the integrity of leads, contacts, conductors, electrodes, and sensing circuitry used to sense the evoked signals may be inferred from sensing integrity.

To the extent contacts, electrodes, conductors, or circuitry are used to sense other signals and/or deliver stimulation, the evaluation of sensing integrity for evoked signals also may provide an indication of reliability of stimulation and other sensing functions performed by the IMD or lead electronics. For example, evaluation of sensing integrity with respect to evoked signals may indicate integrity of other sensing functions, such as sensing of intrinsic signals, as well as stimulation functions.

The techniques described in this disclosure may be especially useful in evaluating lead integrity as a subset of overall sensing integrity. Overall lead integrity is a function of structural and electrical integrity of the lead body. In some cases, lead integrity may be analyzed based on lead impedance measurements. However, adverse effects of lead-related conditions, such as open or short circuits, may be intermittent and difficult to detect based on impedance. Sensing integrity may be evaluated over time on a continuous or periodic basis to develop trend data, which may be effective in inferring lead-related conditions.

Notably, the techniques described in this disclosure make use of evoked signals generated in response to delivery of stimulation, instead of, or in addition to, intrinsic signals. In this manner, there may be no need to manage feature interactions in an IMD to force sensed events to occur so that an intrinsic event can be measured and trended. Rather, evoked signals, such as cardiac depolarization potentials that follow a capturing pacing pulse, can be sensed through the same lead and vector that would be used for intrinsic sensing.

Further, in some examples, techniques described in this disclosure may allow an IMD to deliver pacing pulses via one or more stimulation vectors while sensing the evoked response via a sensing vector that is different from the stimulation vector. In particular, in some cases, at least some of the electrodes that define the stimulation vector may be different from one or more of the electrodes that define the sensing vector. These vectors may include electrodes on the same lead, or, alternatively, on different leads.

In one example, an IMD may utilize one or more high energy coil electrodes to sense signals evoked by pacing pulses delivered via other electrodes of the IMD. In this manner, the IMD may sense the evoked signals via a sensing vector including at least one high energy coil electrode as a sense electrode. In any case, in this manner, sensing integrity of an IMD may be evaluated while pacing pulses are also being delivered by the IMD.

In some devices, such as cardiac resynchronization therapy (CRT) devices, it is often desirable to maintain ventricular pacing for one-hundred percent or nearly one-hundred percent of the time. In addition to pacing, however, it is desirable to periodically assess sensing integrity, particularly in CRT defibrillators, which generally require intact sensing to support proper operation, e.g., proper synchronization of pulses delivered to right and left ventricles. For purposes of remote follow-up when the patient is not in clinic, providing a sensing trend is desirable to establish confidence in IMD functions.

Instead of forcing intrinsic ventricular sensed events to occur, so that an intrinsic event can be measured and trended, this disclosure presents techniques for utilization of evoked potentials to determine sensing integrity over time. Evoked signals may be measured and trended in the presence of substantially continuous pacing, such as one-hundred percent pacing, to permit evaluation of sensing integrity. Once evoked signal data has been sensed, collected and trended, sensing integrity can be determined in a variety of ways, e.g., by automated or visual inspection of the trend itself, or by automated algorithmic analysis of the data with respect to a variety of characteristics such as percent change in amplitude, minimum amplitude thresholds, maximum amplitude thresholds, average or mean amplitude thresholds, or other characteristics and statistical methods.

Evaluation of sensing integrity based on analysis of evoked response signals may be performed alone or in conjunction with other techniques for evaluation of sensing integrity. For example, in some implementations, evaluation of sensing integrity based on analysis of evoked signals may be performed in combination with evaluation of lead integrity based on impedance measurements or sensing of intrinsic signals. Hence, analysis of evoked response may be used as a sole, primary or secondary indication of sensing integrity.

Sensing and analysis of evoked signals to evaluate sensing integrity may be performed within an IMD in some implementations. As an alternative, the IMD may sense evoked signals and store evoked signal data. An external device such as a programmer, home monitor, handheld programmer or other device may be configured to retrieve and analyze the evoked signal data stored by the IMD to evaluate sensing integrity. Accordingly, various features of the techniques described in this application may be performed within a single device or a combination of devices that cooperate to evaluate sensing integrity, and thereby identify potential lead-related conditions, sensing circuitry conditions, or the like.

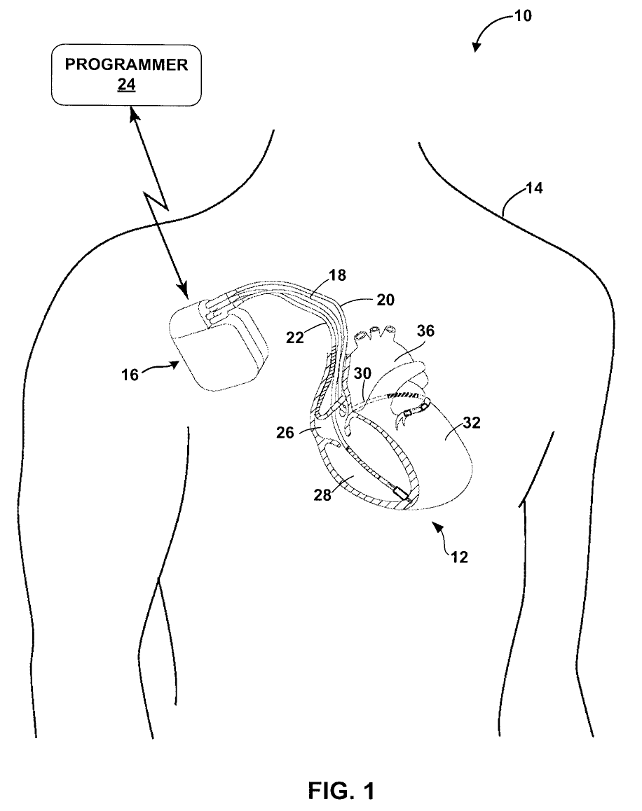

FIG. 1 is a conceptual diagram illustrating an example therapy system 10 that may be used to provide therapy to heart 12 of patient 14. Patient 12 ordinarily, but not necessarily, will be a human. Therapy system 10 includes an IMD 16, which is coupled to leads 18, 20, and 22, and programmer 24. In the example of FIG. 1, IMD 16 may be, for example, an implantable pacemaker, cardioverter, and/or defibrillator that provides electrical stimulation signals to heart 12 via electrodes coupled to one or more of leads 18, 20, and 22. In other applications, IMD 16 may take a variety of forms such as an implantable spinal cord stimulator, gastric stimulator, deep brain stimulator, pelvic floor stimulator, functional electrical stimulator, or the like.

Leads 18, 20, 22 extend into the heart 12 of patient 16 to sense electrical activity of heart 12 and/or deliver electrical stimulation to heart 12. In the example shown in FIG. 1, right ventricular (RV) lead 18 extends through one or more veins (not shown), the superior vena cava (not shown), and right atrium 26, and into right ventricle 28. Left ventricular (LV) coronary sinus lead 20 extends through one or more veins, the vena cava, right atrium 26, and into the coronary sinus 30 to a region adjacent to the free wall of left ventricle 32 of heart 12. Right atrial (RA) lead 22 extends through one or more veins and the vena cava, and into the right atrium 26 of heart 12.

System 10 may sense one or more cardiac signals evoked in response to delivery of electrical stimulation. For example, IMD 16 may deliver electrical stimulation to heart 12 via one or more electrodes on any of implantable leads 18, 20, 22. One or more cardiac signals evoked by the stimulation tissue in response to the electrical stimulation may be sensed via one or more electrodes on any of implantable leads 18, 20, 22. The sensed evoked cardiac signals may be analyzed to evaluate sensing integrity of a sensing module, including leads 18, 20, 22 and electrodes used by the sensing module to sense cardiac signals. Reliability of one or more leads 18, 20, or 22 may be inferred based on the sensing integrity of the sensing module. In some cases, however, sensing integrity issues may be related to circuit or environmental issues, rather than lead integrity issues. In any event, lead integrity may be inferred from sensing integrity in many situations.

IMD 16 may sense electrical signals attendant to the depolarization and repolarization of heart 12 via electrodes (not shown in FIG. 1) coupled to at least one of the leads 18, 20, 22. The sensed electrical signals may be intrinsic signals of heart 12, i.e., depolarization signals naturally produced by the normal function of the cardiac tissue. For purposes of evaluating sensing integrity, in accordance with this disclosure, the sensed signals may be signals evoked by the delivery of electrical stimulation to heart 12, i.e., depolarization signals generated by the cardiac tissue in response to application of a pacing pulse. In some examples, IMD 16 may provide pacing pulses to heart 12 on a continuous basis or in response to the absence of an intrinsic pulse within heart 12.

Various configurations of electrodes used by IMD 16 for sensing and pacing may be unipolar or bipolar. In addition to pacing, IMD 16 may also provide defibrillation therapy and/or cardioversion therapy via electrodes located on at least one of the leads 18, 20, 22 and, more typically, via a combination of one or more elongated coil electrodes and another electrode, such as an electrode carried by a housing associated with IMD 16. The coil electrodes may be high voltage, high energy electrodes for delivery of cardioversion shocks and/or defibrillation shocks. IMD 16 may detect arrhythmia of heart 12, such as fibrillation of ventricles 28 and 32, and deliver defibrillation shock therapy to heart 12 in the form of high energy electrical pulses. In some examples, IMD 16 may be programmed to deliver a progression of therapies, e.g., pulses with increasing energy levels, until a fibrillation of heart 12 is stopped. IMD 16 detects fibrillation employing one or more fibrillation detection techniques known in the art.

In some examples, external programmer 24 may be a handheld computing device, a computer workstation, or a home monitor device. Such devices may be configured to allow for one or more appropriate operations, including but not limited to the remote programming of IMD 16 and/or the remote retrieval of stored data. For example, programmer 24 may include a home monitor device connected to an off-site network device which may communicate with the home monitor device to program IMD 16 and/or retrieve data stored on IMD 16. In some cases, programmer 24 may be configured for wireless access to perform one or more functions, such as, programming of IMD 16, collection of sense data or operational data stored by IMD 16, and/or analysis of the stored data. In this manner, one or more aspects of the disclosure may be performed by a device or user at a location that is remote from the patient.

Further, as a home monitor or handheld device, programmer 24 may be configured to provide one or more types of an alert to a patient and/or physician of sensing reliability based on the evaluation of the sensed signals. For example, programmer may provide an appropriate audible or visual alert to a patient or physician based on an evaluation of the sensed, evoked signals. In some cases, programmer 24 may be connected to an off-site network device to communicate alerts to a user such as a clinician as a function of the evaluation of the sensed signals, and allow a user to properly and timely address any sensing reliability issues associated with IMD 16. Programmer 24 may generally refer to a programmer used in-clinic by a clinician or other caregiver, or a local monitoring and/or programming device co-located with the patient.

Hence, a home monitor, handheld programmer, or other device co-located with the patient may be configured to not only facilitate remote monitoring and programming, but also generate audible, visual, text or graphical alerts or notifications to the patient, or otherwise communicate with the patient or a local caregiver, to indicate a sensing integrity condition that may warrant attention by a remote caregiver such as a clinician. For example, the home monitor may generate a local notification in any of a variety of ways, such as by sounding an alert or light, or presenting a message on a display screen, or send a message to the caregiver and the patient remotely via a network, e.g., via a telephone, email, text message, instant message or the like.

Programmer 24 may include a user interface that receives input from a user. The user interface may include, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. Programmer 24 can additionally or alternatively include a peripheral pointing device, such as a mouse, via which a user may interact with the user interface. In some embodiments, a display of programmer 24 may include a touch screen display, and a user may interact with programmer 24 via the display.

A user, such as a physician, technician, clinician, or other caregiver, may interact with programmer 24 to communicate with IMD 16. For example, the user may interact with programmer 24 to retrieve physiological or diagnostic information from IMD 16, such as data relating to sensed evoked potentials for use in evaluating sensing integrity. A user may also interact with programmer 24 to program IMD 16, e.g., select values for operational parameters of the IMD. Again, such data may be relayed to a remote programmer via a home monitor or other device co-located with the patient.

A user may use programmer 24 to retrieve information from IMD 16 regarding the rhythm of heart 12, trends therein over time, or arrhythmic episodes. The user also may use programmer 24 to retrieve information from IMD 16 regarding other sensed physiological parameters of patient 14, if available, such as intracardiac or intravascular pressure, pulse oximetry, blood perfusion, activity, posture, respiration, or thoracic impedance. As another example, the user may use programmer 24 to retrieve information from IMD 16 regarding the performance or integrity of IMD 16 or other components of system 10, such as leads 18, 20 and 22, or a power source of IMD 16, including data relating to sensed evoked potentials as described in this disclosure.

In some cases, for example, a user may retrieve information regarding the sensed cardiac signals evoked by delivery of electrical stimulation from IMD 16, e.g., using programmer 24. IMD 16 may store the information relating to sensed evoked signals in a raw format, or preprocess the information to provide parametric, morphological, or trend information. For example, IMD 16 may produce and store trend information, such as mean amplitude or other types of information relating to the sensed evoked signals.

In some implementations, IMD 16 may be configured to analyze at least some of the information relating to sensed evoked signals to evaluate sensing integrity. Hence, sensing, processing and analysis of the information may be provided in IMD 16 such that IMD 16 provides some or all of the analysis necessary to evaluate sensing integrity. Alternatively, some of the processing and analysis may be performed by an external device such as programmer 24.

IMD 16 may generate an indication of sensing integrity or, in some cases, an indication of lead integrity. IMD 16 may store such an indication and/or transmit the indication by wireless telemetry to programmer 24 or another external device. Additionally, or alternatively, IMD 16 may generate an audible or tactile alert for the patient in the event sensing integrity or lead integrity is questionable. In response, the patient may elect to promptly visit the clinic for further evaluation of potential lead-related conditions or other conditions that may alter sensing integrity.

IMD 16 may adjust operation of the sensing and/or stimulation features of the IMD in response to indication of a questionable sensing integrity. For example, IMD 16 may bypass particular combinations of electrodes that may present lead-related conditions and/or use different leads or electrodes for sensing and/or therapy.

In other implementations, IMD 16 may simply store information relating to sensed evoked potentials, either in a raw or preprocessed format, and leave significant analysis of such information to be performed by programmer 24 or another external device. In this case, programmer 24 may retrieve information from IMD 16 for purposes of archival, processing and analysis in order to evaluate sensing integrity.

The evoked potential information obtained from IMD 16 may be displayed to a user via a user interface of programmer 24 or any other suitable device for displaying such data to a user. A user may analyze the retrieved information, e.g., by visual inspection, and evaluate sensing integrity. In turn, based on the evaluation of sensing integrity, the user may evaluate reliability of one or more implantable leads 18, 22, and 20, sensing electronics, or other features of IMD 16.

In general, the user may use programmer 24 to program a therapy progression, select electrodes used to deliver defibrillation pulses, select waveforms for the defibrillation pulse, or select or configure a fibrillation detection algorithm for IMD 16. The user may also use programmer 24 to program aspects of other therapies provided by IMD 14, such as cardioversion or pacing therapies. In some examples, the user may activate certain features of IMD 16 by entering a single command via programmer 24, such as depression of a single key or combination of keys of a keypad or a single point-and-select action with a pointing device. When sensing integrity appears to be altered, programmer 24 may be used to reconfigure programming of IMD 16 to restore sensing integrity.

IMD 16 and programmer 24 may communicate with one another via wireless telemetry using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the IMD 16 implant site in order to improve the quality or security of communication between IMD 16 and programmer 24.

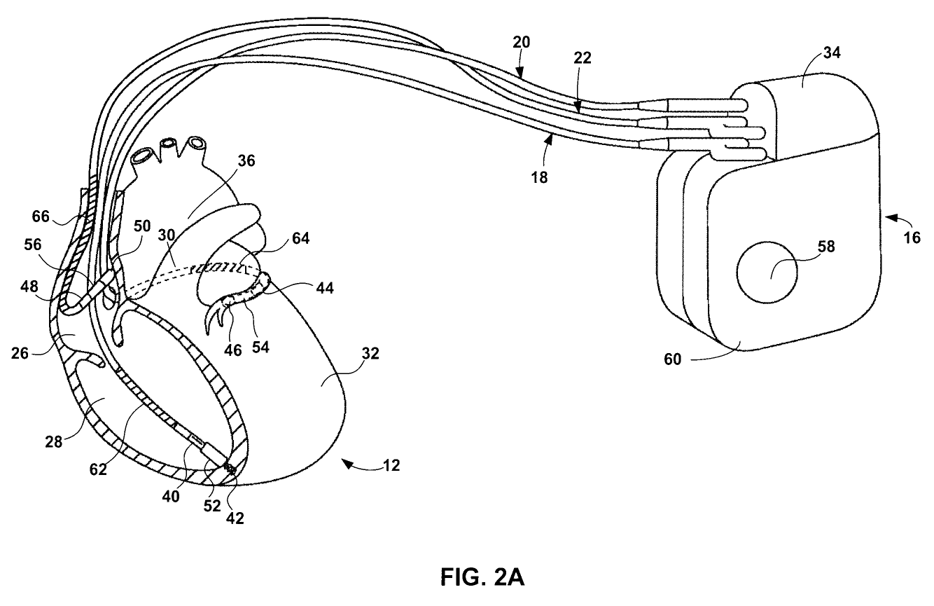

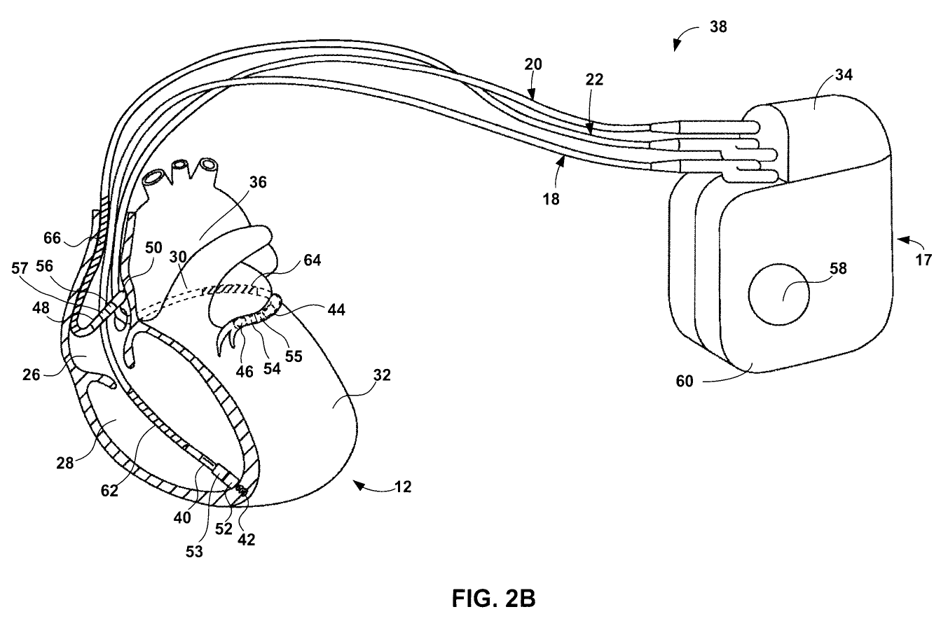

FIG. 2A is a conceptual diagram illustrating IMD 16 and leads 18, 20, and 22 of therapy system 10 in greater detail. Leads 18, 20, 22 may be electrically coupled to an implantable stimulation generator and an implantable sensing module of IMD 16 via connector block 34. The implantable stimulation generator is configured to deliver cardiac pacing stimulation to cardiac tissue via leads 18, 20, 22. In some examples, proximal ends of leads 18, 20, 22 may include electrical contacts that electrically couple to respective electrical contacts within connector block 34. In addition, in some examples, leads 18, 20, 22 may be mechanically coupled to connector block 34 with the aid of set screws, connection pins or another suitable mechanical coupling mechanism.

Each of the leads 18, 20, 22 includes an elongated insulative lead body carrying a number of concentric coiled conductors separated from one another by tubular insulative sheaths. Bipolar electrodes 40 and 42 are located adjacent to a distal end of lead 18. In addition, bipolar electrodes 44 and 46 are located adjacent to a distal end of lead 20 and bipolar electrodes 48 and 50 are located adjacent to a distal end of lead 22. Electrodes 40, 44 and 48 may take the form of ring electrodes, and electrodes 42, 46 and 50 may take the form of extendable helix tip electrodes mounted retractably within insulative electrode heads 52, 54 and 56, respectively. Each of the electrodes 40, 42, 44, 46, 48 and 50 may be electrically coupled to a respective one of the coiled conductors within the lead body of its associated lead 18, 20, 22, and thereby coupled to respective ones of the electrical contacts on the proximal end of leads 18, 20 and 22.

Electrodes 40, 42, 44, 46, 48 and 50 may sense electrical signals attendant to the depolarization and repolarization of heart 12. These sensed signals may include those evoked by the delivering of electrical stimulation to heart 12 of patient 14. This disclosure describes techniques to support evaluation of sensing integrity of the implantable sensing module of IMD 16 based on analysis of stored data, such as trend data, relating to sensed evoked cardiac signals.

The electrical signals are conducted to IMD 16 via the respective leads 18, 20, 22. In some examples, IMD 16 also delivers pacing pulses via electrodes 40, 42, 44, 46, 48 and 50 to cause depolarization of cardiac tissue of heart 12. In some examples, as illustrated in FIG. 2A, IMD 16 includes one or more housing electrodes, such as housing electrode 58, which may be formed integrally with an outer surface of hermetically-sealed housing 60 of IMD 16 or otherwise coupled to housing 60.

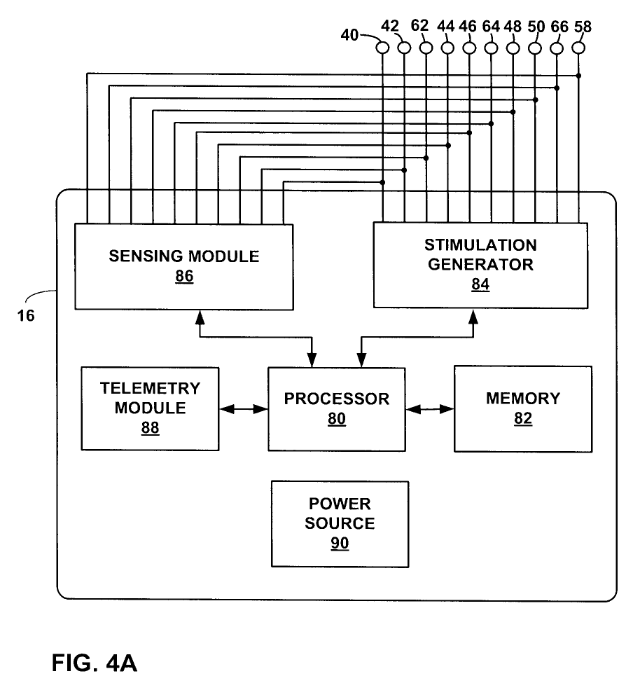

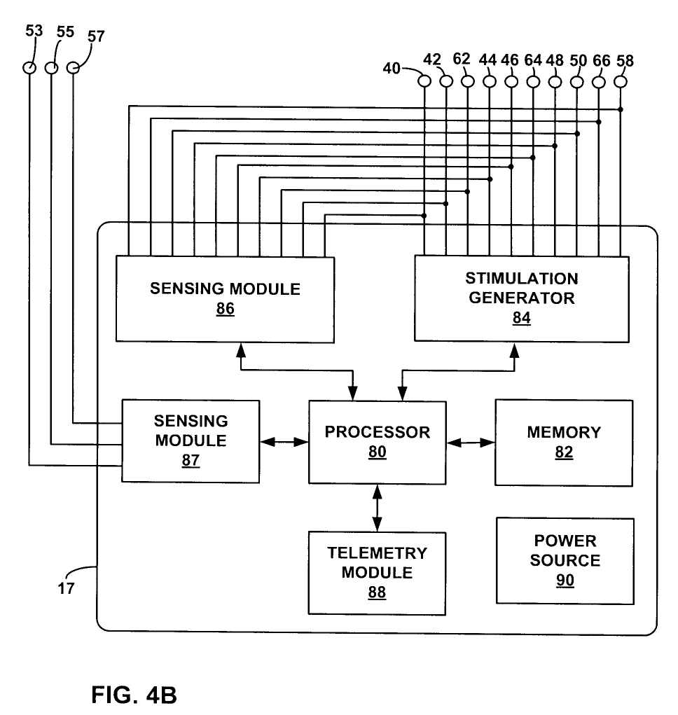

In some examples, housing electrode 58 is defined by an uninsulated portion of an outward facing portion of housing 60 of IMD 16. Other divisions between insulated and uninsulated portions of housing 60 may be employed to define two or more housing electrodes. In some examples, housing electrode 58 comprises substantially all of housing 60. Any of the electrodes 40, 42, 44, 46, 48 and 50 may be used for unipolar sensing or pacing in combination with housing electrode 58. As described in further detail with reference to FIG. 4A, implantable housing 60 may enclose an implantable stimulation generator that generates cardiac pacing pulses and/or cardioversion-defibrillation shocks, as well as a sensing module for monitoring the heart rhythm of the patient.

Leads 18, 20, 22 may include elongated electrodes 62, 64, 66, respectively, which may take the form of a coil. IMD 16 may deliver defibrillation shocks to heart 12 via any combination of elongated, coil electrodes 62, 64, 66, and housing electrode 58. Electrodes 58, 62, 64, 66 may also be used to deliver cardioversion shocks to heart 12. Coil electrodes 62, 64, 66 and other electrodes may be fabricated from any suitable electrically conductive material, such as platinum, platinum alloy or other materials known to be usable in implantable electrodes. In some examples, any of elongated electrodes 62, 64, and 66 may also be used to sense cardiac signals, e.g., cardiac signals evoked by the delivery of electrical stimulation to heart 12. For example, any of elongated electrodes 62, 64, and 66 may be utilized to sense cardiac signals evoked pacing stimulation delivered via any of electrodes 40, 42, 44, 46, 48, and 50.

The configuration of therapy system 10 illustrated in FIGS. 1 and 2A is merely one example. In other examples, a therapy system may include epicardial leads and/or patch electrodes instead of or in addition to the transvenous leads 18, 20, 22 illustrated in FIG. 1. In addition, in some cases, IMD 16 may include one or more subcutaneous electrodes for sensing and delivery of pacing pulses and/or cardioversion-defibrillation energy. Further, IMD 16 need not be fully implanted within patient 14. In examples in which IMD 16 is not fully implanted in patient 14, IMD 16 may deliver defibrillation pulses and other therapies to heart 12 via percutaneous leads that extend through the skin of patient 14 to a variety of positions within or outside of heart 12.

Further, in some examples, an IMD may include one or more sensing devices configured to sense signals associated with one or more parameters, e.g., parameters associated with a patient and/or the therapy being delivered to the patient by the respective therapy system, without utilizing one or more of electrodes 40, 42, 44, 46, 48, 50, 62, 64, and 66 on leads 18, 20, and 22. Accordingly, such sensing devices may be utilized to sense such signals in addition to the signals sensed via one or more of electrodes 40, 42, 44, 46, 48, 50, 62, 64, and 66 on leads 18, 20, and 22. In some cases, the signals sensed by such types of sensing devices may be utilized by a therapy system to monitor the condition of a patient, either alone or in conjunction with the signals obtained via sense electrodes, so that appropriate therapy may be delivered to a patient.

FIG. 2B is a conceptual diagram illustrating a portion of another example therapy system 38 similar to the portion of therapy system 10 illustrated in FIG. 2A, except that leads, 18, 20, and 22 of IMD 17 further includes lead-based, electronic sensing devices 53, 55, and 57, respectively. As illustrated by FIG. 2B, electronic sensing devices 53, 55, and 57 may be located adjacent to the distal end of leads 18, 20, and 22, between electrodes 40, 44 and 48, respectively. However, the location of electronic sensing devices 53, 55, and 57 on leads 18, 20, and 22 is not limited to that illustrated in FIG. 2B, but instead may be located at any suitable location along leads 18, 20, and 22, and, more generally, any suitable location on IMD 17. Although each of leads 18, 20, and 22 of IMD 17 includes an individual electronic sensing device, in other examples, less than all of leads on an IMD may include such an electronic sensing device.

In the example illustrated in FIG. 2B, lead-based, electronic sensing devices 53, 55, and 57 may be one or more of the types of electronic sensing devices previously described, e.g., oxygen sensors, accelerometer sensors, pressure sensors, and ultrasound sensors. In this manner, signals associated with one or more parameters may be sensed by IMD 17, e.g., oxygen concentration, tissue perfusion, activity, posture, motion, blood pressure, motion, or the like, in addition to the signals sensed by one or more of electrodes 40, 42, 44, 46, 48, 50, 62, 64, and 66 on leads 18, 20, and 22.

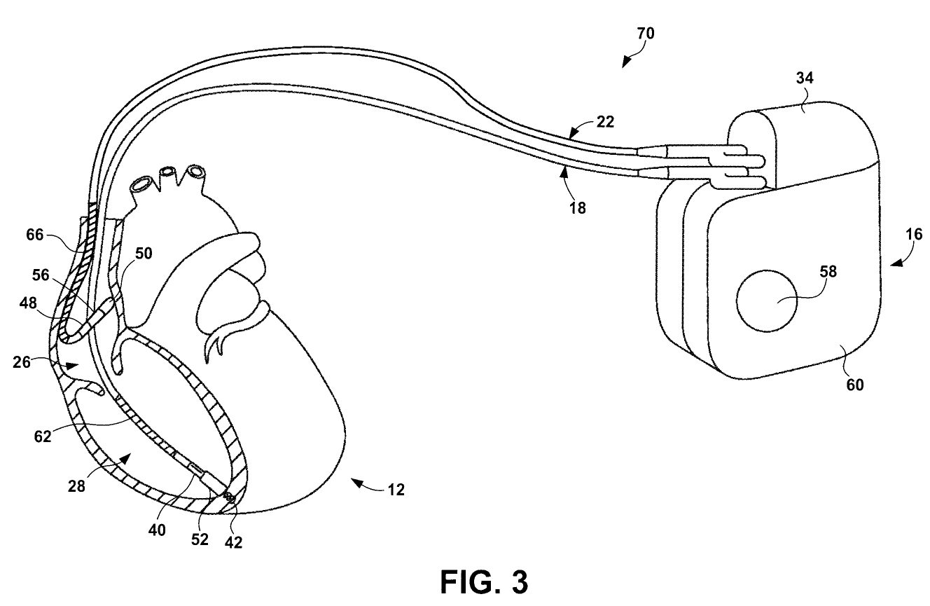

In addition, in other examples, a therapy system may include any suitable number of leads coupled to IMD 16, and each of the leads may extend to any location within or proximate to heart 12. For example, other examples of therapy systems may include three transvenous leads located as illustrated in FIGS. 1 and 2A, and an additional lead located within or proximate to left atrium 36. As another example, a therapy system may include a single lead that extends from IMD 16 into right atrium 26 or right ventricle 28, or two leads that extend into a respective one of the right ventricle 26 and right atrium 28. An example of this type of therapy system is shown in FIG. 3.

FIG. 3 is a conceptual diagram illustrating another therapy system 70, which is similar to therapy system 10 of FIGS. 1 and 2A, but includes two leads 18, 22, rather than three leads. Leads 18, 22 are implanted within right ventricle 28 and right atrium 26, respectively. Therapy system 70 shown in FIG. 3 may be useful for providing cardioversion-defibrillation shocks and pacing pulses to heart 12.

FIG. 4A is a functional block diagram of one example of IMD 16. As shown in FIG. 4A, IMD 16 may include a processor 80, memory 82, stimulation generator 84, sensing module 86, telemetry module 88, and power source 90. Memory 82 includes computer-readable instructions that, when executed by processor 80, cause IMD 16 and processor 80 to perform various functions attributed to IMD 16 and processor 80 in this disclosure. Memory 82 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Memory 82 may be a single memory module, or a combination of multiple memory modules including combinations of one or more types of memory as described above.

In some examples, memory 82 may store information relating to sensed cardiac signals evoked by the delivery of electrical stimulation, such as information indicative of the raw sensed signals, parametric data, morphological data, trend data, or the like. Such information may be utilized to evaluate sense integrity and, in turn, the reliability of one or more implantable leads 18, 20, and 22 of IMD 16, or the reliability of other components.

Processor 80 may include one or more of a microprocessor, a controller, a digital signal processor (DSP), an application specific integrated circuit (ASIC), a field-programmable gate array (FPGA), or equivalent discrete or integrated logic circuitry. In some examples, processor 80 may include multiple components, such as any combination of one or more microprocessors, one or more controllers, one or more DSPs, one or more ASICs, or one or more FPGAs, as well as other discrete or integrated logic circuitry. Accordingly, processor 80 may refer to a single processing and control unit, or a combination of processing and control units, in whatever form or combination, useful in controlling the functionality of IMD 16.

The functions attributed to processor 80 in this disclosure may be realized by software, firmware, hardware or any combination thereof. Implantable stimulation generator 84 is configured to deliver cardiac pacing stimulation to cardiac tissue. Processor 80 controls stimulation generator 84 to deliver stimulation therapy to heart 12 according to a selected one or more of therapy programs, which may be stored in memory 82. Specifically, processor 44 may control stimulation generator 84 to deliver electrical pulses with amplitudes, pulse widths, frequency, or electrode polarities specified by the selected therapy programs.

As shown in FIG. 4A, stimulation generator 84 is electrically coupled to electrodes 40, 42, 44, 46, 48, 50, 58, 62, 64, and 66, e.g., via conductors of the respective lead 18, 20, 22, or, in the case of housing electrode 58, via an electrical conductor disposed within housing 60 of IMD 16. Stimulation generator 84 is configured to generate and deliver electrical stimulation therapy to heart 12. For example, stimulation generator 84 may deliver defibrillation shocks to heart 12 via at least two electrodes 58, 62, 64, 66. Stimulation generator 84 may deliver pacing pulses via ring electrodes 40, 44, 48 coupled to leads 18, 20, and 22, respectively, and/or helical electrodes 42, 46, and 50 of leads 18, 20, and 22, respectively.

In some examples, stimulation generator 84 delivers pacing, cardioversion, or defibrillation stimulation in the form of electrical pulses or shocks. In other examples, stimulation generator may deliver one or more of these types of stimulation in the form of other signals, such as sine waves, square waves, or other substantially continuous time signals.

Stimulation generator 84 may include a switch module and processor 80 may use the switch module to select, e.g., via a data/address bus, electrodes to be used to deliver cardioversion-defibrillation shocks or pacing pulses. The switch module may include a switch array, switch matrix, multiplexer, or any other type of switching device suitable for selectively coupling stimulation energy to selected electrodes.

Sensing module 86 may be configured to monitor one or more signals from at least one of electrodes 40, 42, 44, 46, 48, 50, 58, 62, 64 or 66 in order to monitor electrical activity of heart 12, e.g., via electrogram (EGM) signals. Signals sensed via a particular electrode may be referred to another electrode on a lead or an electrode on the housing of IMD 16. Sensing module 86 may also include a switch module to select which of the available electrodes, or which pairs or combinations of electrodes, are used to sense the heart activity. This disclosure describes techniques to support evaluation of sensing integrity of the implantable sensing module 86 of IMD 16 based on analysis of stored data relating to sensed evoked signals. For purposes of sensing integrity, sensing module 86 may be considered to include leads 18, 20, 22, which may provide sense electrodes for use in sensing evoked cardiac signals and other signals.

In some examples, processor 80 may select the electrodes that function as sense electrodes via the switch module within sensing module 86, e.g., by providing signals via a data/address bus. Sensing module 86, in some cases, may be configured specifically for the purpose of sensing evoked signals. For example, sensing module 86 may include any combination of one or more different types of amplifiers configured for one or more specific types of sensing. Sense module 86 may include a bank of different sense amplifiers specific to one or more sensed signals. For example, sense module 86 may include one or more of a separate sensed LV signal amplifiers, sensed RV signal amplifiers, sensed atrial signal amplifiers, or sensed evoked signal amplifiers. The one or more separate amplifiers may be configured or programmable to perform one or more types of sensing.

Signals produced by the sense amplifiers may be converted from analog signals to digital signals by analog-to-digital converters (ADCs) provided by sensing module 86. The digital signals may be stored in memory for analysis on-board the IMD 16 or remote analysis by a programmer 24 or other device. Sensing module 86 may include a digital signal processor (DSP) that implements any of a variety of digital signal processing features such as digital amplifiers, digital filters, and the like. In general, the DSP may process the received signals to extract information useful in the evaluation of a signal for purposes of evaluating sensing reliability. Examples of such information include but are not limited to waveform characteristics such as signal amplitude and frequency, and other morphological characteristics, the relative area under a curve created by a sensed signal, the slope of a curve created by a sensed signal, inflection points of a curve created by a sensed signal.

Hence, the DSP may be configured to apply any of a variety of signal processing filters and algorithms to extract desired data from the sense signals, and also apply various statistical analysis algorithms to determine whether a signal is indicative of positive or negative sensing integrity. Negative sensing integrity may indicate a sensing integrity condition. Again, the signal relates to the signal evoked by cardiac tissue in response to delivery of electrical stimulation such as a pacing pulse, and may be an electrical signal sensed via sense electrodes, or other signals such as signals sensed incident to an evoked signal by oxygen sensors, accelerometer sensors, pressure sensors, ultrasound sensors, or other types of sensors.

In some examples, sensing module 86 includes one or more sensing channels, each of which may comprise an amplifier, as described above. In response to the signals from processor 80, the switch module within sensing module 86 may couple the outputs from the selected electrodes to one of the sensing channels.

For more general sensing, one channel of sensing module 86 may include an R-wave amplifier that receives signals from electrodes 40 and 42, which are used for pacing and sensing in right ventricle 28 of heart 12. Another channel may include another R-wave amplifier that receives signals from electrodes 44 and 46, which are used for pacing and sensing proximate to left ventricle 32 of heart 12. In some examples, the R-wave amplifiers may include an automatic gain controlled (AGC) amplifier that provides an adjustable sensing threshold as a function of the R-wave amplitude of the heart rhythm.

In addition, one channel of sensing module 86 may include a P-wave amplifier that receives signals from electrodes 48 and 50, which are used for pacing and sensing in right atrium 26 of heart 12. In some examples, the P-wave amplifier may include an automatic gain controlled amplifier that provides an adjustable sensing threshold as a function of the measured P-wave amplitude of the heart rhythm.

Examples of R-wave and P-wave amplifiers are described in U.S. Pat. No. 5,117,824 to Keimel et al., which issued on Jun. 2, 1992 and is entitled, “APPARATUS FOR MONITORING ELECTRICAL PHYSIOLOGIC SIGNALS,” and is incorporated herein by reference in its entirety. Other amplifiers may also be used. Furthermore, in some examples, one or more of the sensing channels of sensing module 84 may be selectively coupled to housing electrode 58, or elongated electrodes 62, 64, or 66, with or instead of one or more of electrodes 40, 42, 44, 46, 48 or 50, e.g., for unipolar sensing of R-waves or P-waves in any of chambers 26, 28, or 32 of heart 12.

In some examples, sensing module 86 includes a channel that comprises an amplifier with a relatively wider pass band than the R-wave or P-wave amplifiers. Signals from the selected sensing electrodes that are selected for coupling to this wide-band amplifier may be provided to a multiplexer, and thereafter converted to multi-bit digital signals by an analog-to-digital converter (ADC), as described above, for storage in memory 82 as an electrogram (EGM). In some examples, the storage of such EGMs in memory 82 may be under the control of a direct memory access (DMA) circuit. Processor 80 may employ digital signal analysis techniques to characterize the digitized signals stored in memory 82 to detect and classify the patient's heart rhythm from the electrical signals. Processor 80 may detect and classify the patient's heart rhythm, e.g., in terms of signal morphology, by employing any of the numerous signal processing methodologies known in the art.

To generate and deliver pacing pulses to heart 12, processor 80 may include a pacer timing and control module, which may be embodied as hardware, firmware, software, or any combination thereof. The pacer timing and control module may comprise a dedicated hardware circuit, such as an ASIC, separate from other processor 80 components, such as a microprocessor, or a software module executed by a component of processor 80, which may be a microprocessor or ASIC.

The pacer timing and control module may include programmable counters which control the basic time intervals associated with DDD, VVI, DVI, VDD, AAI, DDI, DDDR, VVIR, DVIR, VDDR, AAIR, DDIR and other modes of single and dual chamber pacing. In the aforementioned pacing modes, “D” may indicate dual chamber, “V” may indicate a ventricle, “I” may indicate inhibited pacing (e.g., no pacing), and “A” may indicate an atrium. The first letter in the pacing mode may indicate the chamber that is paced, the second letter may indicate the chamber that is sensed, and the third letter may indicate the chamber in which the response to sensing is provided.

Intervals defined by the pacer timing and control module within processor 80 may include atrial and ventricular pacing escape intervals, refractory periods during which sensed P-waves and R-waves are ineffective to restart timing of the escape intervals, and the pulse widths of the pacing pulses. As another example, the pace timing and control module may define a blanking period, and provide signals sensing module 86 to blank one or more channels, e.g., amplifiers, for a period during and after delivery of electrical stimulation to heart 12. The durations of these intervals may be determined by processor 80 in response to stored data in memory 82. The pacer timing and control module of processor 80 may also determine the amplitude of the cardiac pacing pulses.

During pacing, escape interval counters within the pacer timing/control module of processor 80 may be reset upon sensing of R-waves and P-waves. Stimulation generator 84 may include pacer output circuits that are coupled, e.g., selectively by a switching module, to any combination of electrodes 40, 42, 44, 46, 48, 50, 58, 62, or 66 appropriate for delivery of a bipolar or unipolar pacing pulse to one of the chambers of heart 12. Processor 80 may reset the escape interval counters upon the generation of pacing pulses by stimulation generator 84, and thereby control the basic timing of cardiac pacing functions, including anti-tachyarrhythmia pacing.

The value of the count present in the escape interval counters when reset by sensed R-waves and P-waves may be used by processor 80 to measure the durations of R-R intervals, P-P intervals, P-R intervals and R-P intervals, which are measurements that may be stored in memory 82. Processor 80 may use the count in the interval counters to detect an arrhythmia event, such as ventricular fibrillation or ventricular tachycardia.

In some examples, processor 80 may operate as an interrupt-driven device, and may be responsive to interrupts from pacer timing and control module, where the interrupts may correspond to the occurrences of sensed P-waves and R-waves and the generation of cardiac pacing pulses. Any necessary mathematical calculations to be performed by processor 80 and any updating of the values or intervals controlled by the pacer timing and control module of processor 80 may take place following such interrupts. A portion of memory 82 may be configured as a plurality of recirculating buffers, capable of holding series of measured intervals, which may be analyzed by processor 80 in response to the occurrence of a pace or sense interrupt to determine whether the patient's heart 12 is presently exhibiting atrial or ventricular tachyarrhythmia.

In some examples, an arrhythmia detection method may include any suitable tachyarrhythmia detection algorithms. In one example, processor 80 may utilize all or a subset of the rule-based detection methods described in U.S. Pat. No. 5,545,186 to Olson et al., entitled, “PRIORITIZED RULE BASED METHOD AND APPARATUS FOR DIAGNOSIS AND GREATMENT OF ARRHYTHMIAS,” which issued on Aug. 13, 1996, or in U.S. Pat. No. 5,755,736 to Gillberg et al., entitled, “PRIORITIZED RULE BASED METHOD AND APPARATUS FOR DIAGNOSIS AND GREATMENT OF ARRHYTHMIAS,” which issued on May 26, 1998. U.S. Pat. No. 5,545,186 to Olson et al. U.S. Pat. No. 5,755,736 to Gillberg et al. are incorporated herein by reference in their entireties. However, other arrhythmia detection methodologies may also be employed by processor 80 in other examples.

In the event that processor 80 detects an atrial or ventricular tachyarrhythmia based on signals from sensing module 86, and an anti-tachyarrhythmia pacing regimen is desired, timing intervals for controlling the generation of anti-tachyarrhythmia pacing therapies by stimulation generator 84 may be loaded by processor 80 into the pacer timing and control module to control the operation of the escape interval counters therein and to define refractory periods during which detection of R-waves and P-waves is ineffective to restart the escape interval counters.

If IMD 16 is configured to generate and deliver cardioversion or defibrillation shocks to heart 12, stimulation generator 84 may include a high voltage charge circuit and a high voltage output circuit. Lower voltage charge and output circuits may be use for generation and delivery of pacing pulses. In the event that generation of a cardioversion or defibrillation pulse is required, processor 80 may employ the escape interval counter to control timing of such cardioversion and defibrillation pulses, as well as associated refractory periods.

In response to the detection of atrial or ventricular fibrillation or tachyarrhythmia requiring a cardioversion pulse, processor 80 may activate a cardioversion/defibrillation control module of processor 80, which may, like the pacer timing and control module, be a hardware component of processor 80 and/or a firmware or software module executed by one or more hardware components of processor 80. The cardioversion/defibrillation control module may initiate charging of the high voltage capacitors of the high voltage charge circuit of stimulation generator 84 under control of a high voltage charging control line.

Processor 80 may monitor the voltage on the high voltage capacitor, e.g., via a voltage charging and potential (VCAP) line. In response to the voltage on the high voltage capacitor reaching a predetermined value set by processor 80, processor 80 may generate a logic signal that terminates charging. Thereafter, timing of the delivery of the defibrillation or cardioversion pulse by stimulation generator 84 is controlled by the cardioversion-defibrillation control module of processor 80. Following delivery of the fibrillation or tachycardia therapy, processor 80 may return stimulation generator 84 to a cardiac pacing function and await the next successive interrupt due to pacing or the occurrence of a sensed atrial or ventricular intrinsic depolarization.

Stimulation generator 84 may deliver cardioversion or defibrillation pulses with the aid of an output circuit that determines whether a monophasic or biphasic pulse is delivered, whether housing electrode 58 serves as cathode or anode, and which electrodes are involved in delivery of the cardioversion or defibrillation pulses. Such functionality may be provided by one or more switches or a switching module of stimulation generator 84.

Telemetry module 88 includes any suitable hardware, firmware, software or any combination thereof for communicating with another device, such as programmer 24 (FIG. 1). Under the control of processor 80, telemetry module 88 may receive downlink telemetry from and send uplink telemetry to programmer 24 with the aid of an antenna, which may be internal and/or external. Processor 80 may provide the data to be uplinked to programmer 24 and the control signals for the telemetry circuit within telemetry module 88, e.g., via an address/data bus. In some examples, telemetry module 88 may provide received data to processor 80 via a multiplexer.

In some examples, processor 80 may transmit atrial and ventricular heart signals (e.g., electrocardiogram signals) produced by atrial and ventricular sense amp circuits within sensing module 86 to programmer 24. Programmer 24 may interrogate IMD 16 to receive the heart signals. Processor 80 may store heart signals within memory 82, and retrieve stored heart signals from memory 82. Processor 80 may also generate and store marker codes indicative of different cardiac events that sensing module 86 detects, and transmit the marker codes to programmer 24. An example pacemaker with marker-channel capability is described in U.S. Pat. No. 4,374,382 to Markowitz, entitled, “MARKER CHANNEL TELEMETRY SYSTEM FOR A MEDICAL DEVICE,” which issued on Feb. 15, 1983 and is incorporated herein by reference in its entirety.

The various components of IMD 16 may be coupled to power source 90, which may include a rechargeable or non-rechargeable battery and associated electronics for converting or conditioning the battery voltage and/or current to produce an operational power level or levels for the IMD. A non-rechargeable battery may be selected to last for several years, while a rechargeable battery may be inductively charged from an external device, e.g., on a daily or weekly basis.

FIG. 4B is a functional block diagram of an example of IMD 17 of FIG. 2B, which is similar to IMD 16 except that IMD 17 may also include sensing module 87. As previously described, in the example of FIG. 2B, leads 18, 20, and 22 of IMD 17 include lead-based, electronic sensing devices 53, 55, and 57, which may sense signals associated one or more parameters in addition to the electrical cardiac signals sensed via one or more sense electrodes 40, 42, 44, 46, 48, 50, 62, 64, and 66. In some cases, as shown in FIG. 4B, IMD 17 may include sensing module 87, in addition to sensing module 86, to support processing or signals sensed by sensing device 53, 55, 57. In some implementations, sensing module 87 may be integrated with sensing module 86 or share some common hardware, firmware or software with sensing module 86. For example, in some cases, sensing modules 86 and 87 may be configured to share a common DSP and memory. As indicated, sensing module 87 may be configured to monitor one or more signals from at least one of sensing devices 53, 55, and 57 in order to monitor the parameter associated with the respective type of sensing device. The structure and function of sensing module 87 may be substantially similar to sensing module 86, but be configured to process signals from sensing devices 53, 55 and 57. Signals generated by devices 53, 55 and 57 and processed by sensing module 87 may be generated coincident with an evoked response generated in response to electrical stimulation delivered to cardiac tissue.

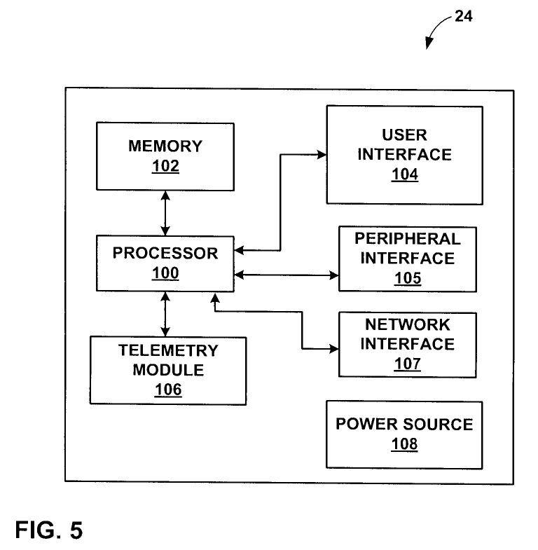

FIG. 5 is block diagram of an example programmer 24. As shown in FIG. 5, programmer 24 includes processor 100, memory 102, user interface 104, peripheral interface 105, telemetry module 106, network interface 107, and power source 108. Programmer 24 may be a dedicated hardware device with dedicated software for programming of IMD 16. Alternatively, programmer 24 may be an off-the-shelf computing device running an application that enables programmer 24 to program IMD 16.

A user such as a medical clinician or other caregiver may use programmer 24 to select therapy programs (e.g., sets of stimulation parameters), generate new therapy programs, modify therapy programs through individual or global adjustments or transmit the new programs to a medical device, such as IMD 16 (FIG. 1). The clinician may interact with programmer 24 via user interface 104, which may include display to present graphical user interface to a user, and a keypad or another mechanism for receiving input from a user. In some embodiments, system 10 may further include a home monitor or patient programmer, e.g., handheld programmer, that is generally co-located with the patient. The home monitor or patient programmer may communicate with IMD 16 via local wireless telemetry and with programmer 24 via network communication.

Processor 100 can take the form one or more microprocessors, DSPs, ASICs, FPGAs, programmable logic circuitry, or the like, or any combination thereof, and the functions attributed to processor 102 herein may be embodied as hardware, firmware, software or any combination thereof. Memory 102 may comprise one or more memory modules or data storage devices, and may store instructions that cause processor 100 to provide the functionality ascribed to programmer 24 herein, and information used by processor 100 to provide the functionality ascribed to programmer 24 herein.

Memory 102 may include any fixed or removable magnetic, optical, or electrical media, such as RAM, ROM, CD-ROM, hard or floppy magnetic disks, EEPROM, or the like. Memory 102 may also include a removable memory portion that may be used to provide memory updates or increases in memory capacities. A removable memory may also allow patient data to be easily transferred to another computing device, or to be removed before programmer 24 is used to program therapy for another patient. Memory 102 may also store information that controls therapy delivery by IMD 16, such as stimulation parameter values, e.g., such as voltage or current amplitude, pulse width, frequency, blanking intervals, escape intervals, or the like.

Programmer 24 may communicate wirelessly with IMD 16, e.g., using RF communication or proximal inductive interaction. This wireless communication may be performed through the use of telemetry module 102, which may be coupled to an internal antenna or an external antenna. An external antenna that is coupled to programmer 24 may correspond to the programming head that may be placed over heart 12, as described above with reference to FIG. 1. Telemetry module 102 may be similar to telemetry module 88 of IMD 16 (FIG. 4A).

Telemetry module 102 may also be configured to communicate with another computing device via wireless communication techniques, or direct communication through a wired connection. Examples of local wireless communication techniques that may be employed to facilitate communication between programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with programmer 24 without needing to establish a secure wireless connection.

Programmer 24 may also include peripheral interface 105 to connect to one or more peripheral devices. For example, peripheral interface 105 may include one or more suitable peripheral interface controllers, e.g., a UBS controller or the like, that allows programmer 24 to connect with a desired peripheral device, e.g., an external memory storage medium.

Programmer 24 may also be configured to communicate with one or more network devices via network interface 107. For example, network interface 107 may include one or more suitable network interface controllers, e.g., an Ethernet port or the like, that allows programmer 24 to connect with a desired network device, e.g., a network server.

Power source 108 delivers operating power to the components of programmer 24. Power source 108 may include a battery and a power generation circuit to produce the operating power. In some embodiments, the battery may be rechargeable to allow extended operation. Recharging may be accomplished by electrically coupling power source 108 to a cradle or plug that is connected to an alternating current (AC) outlet. In addition or alternatively, recharging may be accomplished through proximal inductive interaction between an external charger and an inductive charging coil within programmer 24.

In other embodiments, conventional batteries (e.g., nickel cadmium or lithium ion batteries) may be used. In addition, programmer 24 may be directly coupled to an alternating current outlet to power programmer 24. Power source 104 may include circuitry to monitor power remaining within a battery. In this manner, user interface 104 may provide a current battery level indicator or low battery level indicator when the battery needs to be replaced or recharged. In some cases, power source 108 may be capable of estimating the remaining time of operation using the current battery.

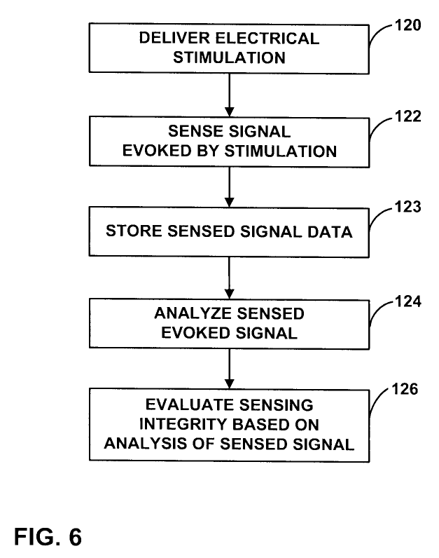

As previously described, examples of the disclosure may utilize cardiac signals evoked by delivery of electrical stimulation to evaluate sensing integrity and monitor the reliability of sensing module 86 and one or more implantable leads or other components associated with sensing module 86 of an IMD. FIG. 6 is a flowchart illustrating an example technique according to an aspect of the present disclosure. For purposes of illustration, the example technique will be generally described with respect to therapy system 10 of FIGS. 1, 2, 4 and 5. However, such a technique is not limited to systems with such configurations but instead may be utilized in any system for which the technique may be suitably applied.

As shown in FIG. 6, IMD 16 delivers electrical stimulation to heart 12 of patient 14 (120). The electrical stimulation may be pacing pulses delivered at regular intervals or sensed intervals. For example, IMD 16 may deliver pacing pulses when an intrinsic pulse is not detected within a prescribed time interval. IMD 16 senses cardiac signals evoked by the cardiac tissue in response to the delivered electrical stimulation (122). IMD 16 may store data representing the sensed evoked signal (123), or other data relating to the sensed evoked signal. The sensed cardiac signal evoked in response to the delivery of electrical stimulation (122) may be analyzed (124), e.g., using processor 80 of IMD 16 or a DSP in sensing module 86, processor 100 of programmer 24, processing hardware associated with another external device, or a combination of processing hardware within IMD 16, programmer 24, or another device.

Based on the analysis of the sensed evoked signal (124), sensing integrity of sensing module 86 may be evaluated (126). Evaluation of sensing integrity may include evaluation of overall sensing integrity, in terms of accurate and reliable sensing of evoked signals, and/or evaluation of lead integrity. In some cases, functional and/or structural integrity of one or more leads 18, 20, 22 may be inferred from sensing integrity. In this manner, the integrity of one or more implantable leads 18, 20, and 22 of IMD 16 may be monitored using one or more cardiac signals evoked by the delivery of electrical stimulation to heart 12.

In general, the electrical stimulation delivered to heart 12 (120) may be generated by stimulation generator 84 and delivered to heart 12 via one or more of the respective electrodes 42, 44, 46, 48, 50, 62, 64, and 66 on leads 18, 20, 22. The electrical stimulation delivered by IMD 16 may be delivered for any suitable purpose, such as described previously, and may be in the form of pacing, defibrillation, or cardioversion stimulation, as described previously. As examples, electrodes 40 and 42 on lead 18 may be utilized to deliver electrical bipolar pacing stimulation to RV 28 of heart 12, or electrode 62 may be utilized to deliver unipolar defibrillation stimulation to RV 28 of heart 12 in conjunction with can electrode 58. However, the electrical stimulation is not limited to being delivered via one or more of electrodes 40, 42, and 62 on lead 18, but may also be delivered via electrodes 44, 46, and 64, on lead 20, or electrodes 48, 50, and 66 on lead 22 in any of a variety of unipolar or bipolar combinations.

Furthermore, the delivery of bipolar electrical stimulation may not be limited to combinations of electrodes contained on the same lead. Instead, in some examples, IMD 16 may be configured to deliver bipolar electrical stimulation via any of electrodes 40, 42, 44, 46, 48, 50, 62, 64, and 66 of leads 18, 20, and 22, in which case the electrodes utilized to deliver the stimulation are on separate leads. For example, bipolar electrical stimulation could be delivered to heart 12 via electrodes 42 and 44 of leads 18 and 20, respectively. In addition, various unipolar combinations may be realized by combinations of electrodes 40, 42, 44, 46, 48, 50, 62, 64, and 66 with can electrode 58 of IMD housing 60.

In any case, one or more cardiac signals may be evoked by tissue of heart 12 upon depolarization in response to the electrical stimulation delivered to heart 12 (120). System 10 may be configured to sense the cardiac signals evoked by delivered electrical stimulation (122), e.g., using sensing module 86 of IMD 16. Sensing a cardiac signal evoked by delivery of electrical stimulation to the heart (122) may include sensing the evoked cardiac potential signal, i.e., the cardiac depolarization that follows a capturing electrical stimulation pulse or shock. Sensing module 86 may be configured as a narrow-band sensing module or wide-band sensing module, and may be configured to sense particular portions of an evoked signal, such as evoked Q, R or S waves, or other wave characteristics of an evoked signal. Such signals may be filtered, rectified and otherwise processed to produce signals that facilitate digital analysis.

In general, IMD 16 may sense the cardiac potential signal evoked by the delivery of electrical stimulation (122) via a bipolar combination one or more electrodes 40, 42, 44, 46, 48, 50, 62, 64, and 66 on any of leads 18, 20, and 22, or a unipolar combination of such electrodes with electrode 58 of IMD can 60, using sensing module 86. In this manner, sensing integrity may be evaluated for multiple electrode combinations and/or leads.

The respective combination of electrodes used to by IMD 16 to sense an evoked signal may be generally referred to as a sensing vector. As previously described, the configurations of electrodes used by IMD 16 for sensing may provide for unipolar or bipolar sensing. For applications of unipolar sensing, the corresponding sensing vector includes electrode 58 (or another can electrode of IMD housing 60) in combination with any of electrodes 40, 42, 44, 46, 48, 50, 62, 64, and 66 to sense the one or more cardiac signals evoked by the delivery of electrical stimulation.

The sensing vector used to sense the evoked signal may correspond to a similar or identical sensing vector used to sense intrinsic signals, e.g., to support pacing. In single-chamber and multi-chamber pacing applications, for example, it may be important to sense an intrinsic signal generated by heart 12 to determine whether to stimulate the heart. For single-chamber pacing, following a previous intrinsic signal or evoked signal, IMD 16 may deliver a pacing pulse if an intrinsic pulse is not sensed within a prescribed time interval. For multi-chamber pacing, such as CRT, IMD 16 may deliver pacing one-hundred percent or nearly one-hundred percent of the time in order to synchronize operation of the right and left ventricles.

In the case of multi-chamber pacing, a pacing pulse may be delivered to the RV if an RV intrinsic pulse is not detected within a time interval. Likewise, an LV pacing pulse may be delivered to the LV if an LV intrinsic pulse is not detected within a time interval following an RV evoked or intrinsic pulse. Alternatively, an LV pacing pulse may be delivered within a prescribed time interval without attempting to sense an intrinsic pulse. The time intervals may be selected to support synchronization and coordination of operation of the RV and LV, e.g., for more efficient pumping operation. In many CRT patients, pacing may be delivered to the RV and LV one-hundred percent of the time. For this reason, it may be difficult to force or permit an intrinsic pulse to be generated for purposes of sensing integrity evaluation. In particular, to force or permit generation of an intrinsic RV or LV pulse, it may be necessary to manage numerous feature interactions within IMD 16

To facilitate evaluation of sensing integrity, in accordance with this disclosure, IMD 16 is configured to sense evoked signals, rather than intrinsic signals. In this manner, sensing integrity information can be obtained using evoked potentials while pacing stimulation is delivered, without the complicated interactions otherwise associated with manipulating device timing and feature functionality to generated sensed intrinsic events. Sensed characteristics of evoked signals may provide an indication of sense integrity, either on an instantaneous basis or as trended over time. Sensing vectors that would be used for intrinsic signal sensing also can be used for evoked signal sensing. In addition, at least some of the sensing vectors may correspond to stimulation vectors, i.e., electrode combinations used for delivery of electrical stimulation. Accordingly, sensing of evoked signals may support analysis of sensing integrity, which also may provide an indication of general lead integrity, e.g., for purposes of evaluating sensing or stimulation reliability.

One or more processors associated with IMD 16, programmer 24 or another external device may analyze one or more characteristics of the sensed signal (124), and evaluate sensing integrity based on the analysis (126). Hence, such one or more processors may be configured or programmed to operate as an evaluation unit to support evaluation of sensing integrity of the implantable sensing module based on analysis of stored data related to the sensed evoked signals.

The evaluation unit may be implantable, forming part of the IMD 16, and be configured to automatically evaluate the sensing integrity. Alternatively, the evaluation unit may form part of the external programmer 24, such as a clinician programmer, home monitor, or the like, wherein the evaluation unit is configured to perform at least one of display of the stored data to a user or automatic evaluation of the sensing integrity.

In each case, if the evaluation unit is configured to perform automatic evaluation of the sensing integrity, the evaluation unit may generate an indication of the sensing integrity to the user. For example, the evaluation unit, whether embodied in IMD 16 or programmer 24, or another device, may provide a notification module configured to generate a notification in the event the evaluation indicates a sensing integrity condition. A sensing integrity condition may refer, in general, to a condition that may alter sensing operation relative to a normal or desired operation.

In some cases, IMD 16 may store wide band raw signal data representing the sensed evoked signals. IMD 16 may store continuous or one-beat snippets of the wide band raw signal waveform, where a beat may refer to a beat of the heart or, more generally, a depolarization of tissue in response to electrical stimulation. Programmer 24 may retrieve the stored data and process the data to generate trend data or other data presenting characteristics for analysis to evaluate sensing integrity. In other cases, IMD 16 may be configured to pre-process the sensed evoked signal data to produce trend data or other data for storage in IMD 16. As an example, IMD 16 may create a template and compare, periodically or beat-to-beat, the degree to which each subsequent beat matches the template. Accordingly, various aspects of processing, analysis and evaluation may be performed in a single device or shared among multiple devices.

For example, sensing, processing, analysis and evaluation may be performed entirely within IMD 16, in which case IMD may generate a sensing integrity indication for communication to or retrieval by programmer 24 or another device. Alternatively, IMD 16 may simply store raw sensed evoked signal data, and programmer 24 or another device may perform the processing, analysis and evaluation of the stored data. As a further alternative, IMD 16 may sense, pre-process and store trend data for retrieval by programmer 24 or another device, in which case the programmer or device may perform the analysis and evaluation of the trend data.

System 10 may be subject to a variety of different implementations, such that various aspects of the techniques described in this disclosure may be performed by processor 80 of IMD 16 pursuant to instructions stored in memory 82 of IMD 16, processor 100 of programmer 24 pursuant to instructions stored in memory 102, a DSP within sensing module 86, or a combination of such devices. In addition, in some implementations, one or more additional devices may receive data obtained from IMD 16 by programmer 24, and perform necessary processing, analysis, and/or evaluation. In each case, one or more processors may be configured or programmed to operate as an evaluation unit to support evaluation of sensing integrity of the implantable sensing module 86, including associated leads 18, 20, 22, based on analysis of stored data related to the sensed evoked signals.

In general, whether performed by IMD 16, programmer 24 or another device, analysis of the sensed evoked cardiac signal may support evaluation of sensing integrity (126) so that potential sensing integrity conditions in sensing module 86, including lead-related conditions of leads 18, 20, 22, may be detected. Any number of suitable techniques may be used to perform this evaluation based on analysis of the sensed cardiac signal. In addition, in some implementations, programmer 24 or another device may be configured to present sensed evoked signal data, such as trend data, for visual inspection by a clinician to support non-automated evaluation of sensing integrity by the clinician.

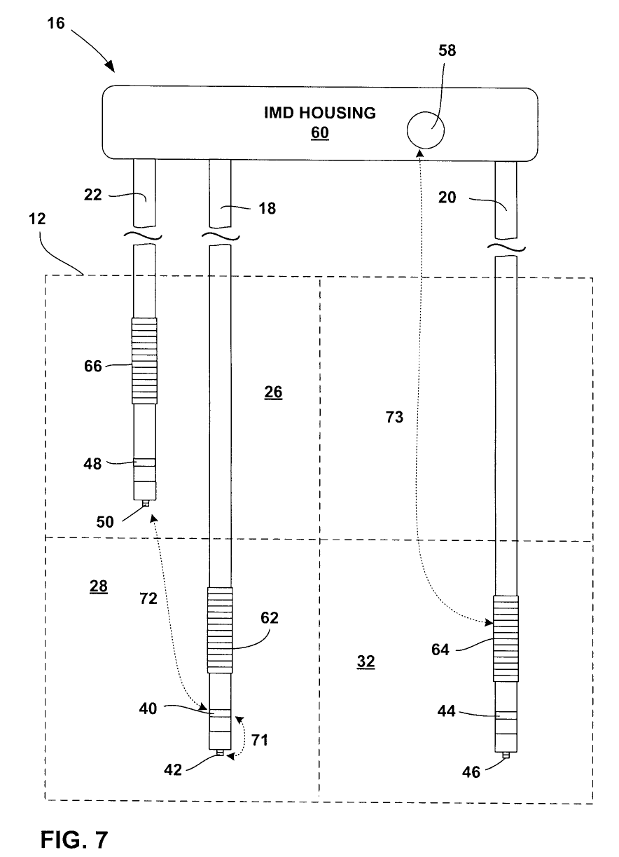

FIG. 7 is a simplified schematic diagram illustrating IMD 16 of therapy system 10. FIG. 7 illustrates various sensing vectors that may be utilized by IMD 16 to evaluate sensing integrity. IMD 16 includes all features as described previously, including implantable leads 18, 20, and 22. The location of lead 18, 20, and 22 with respect to heart 12 is indicated by dashed sections corresponding to RA 26, RV 28, and LV 32. However, the path followed by leads 18, 20, and 22 to IMD housing 60 with respect to the dashed sections indicated by FIG. 7 are not necessarily representative of an actual configuration of an IMD 16 implanted in the heart of a patient.

IMD 16 may utilize a variety of sensing vectors to sense one or more cardiac signals evoked by delivered electrical stimulation. In some examples, a sensing vector may be a single lead sensing vector, i.e., including electrode(s) from only one of leads 18, 20, and 22. For example, IMD 16 may sense an evoked cardiac signal using a bipolar sensing vector including electrodes 40 and 42 of lead 18, indicated by arrow 71. In other examples, a bipolar sensing vector may be multi-lead sensing vector, i.e., including at least two electrodes that are provided on separate implantable leads. For example, IMD 16 may sense an evoked cardiac signal using a sensing vector including electrode 50 of lead 22 and electrode 40 of lead 18, indicated by arrow 72. In still other examples, electrode 58 of IMD housing 60 may be included as an electrode in a unipolar sensing vector, e.g., unipolar sensing vectors including electrode 58 and any one of electrodes 40, 42, 44, 46, 48, 50, 62, 64, and 66. For example, IMD 16 may sense an evoked cardiac signal using a sensing vector including electrode 58 of IMD housing 60 and electrode 64 of lead 20, as indicated by arrow 73.

Furthermore, in some examples, the configuration of respective sensing vectors used by IMD 16 to sense evoked cardiac signals is not limited by the type of electrodes used. In some cases, a sensing vector may include electrodes of the same type, e.g., ring electrodes 40 and 44. Additionally, in some cases, a sensing vector may include electrodes of different types, e.g., a sensing vector including ring electrode 44 and helix tip electrode 46, or ring electrode 44 and elongated coil electrode 64, or can electrode 58 and coil electrode 62 or 64.

In some examples, sensing vectors used by IMD 16 to sense evoked cardiac signals are not limited with respect to the electrode(s) used for delivery of the electrical stimulation that evokes the cardiac signal. For example, if pacing stimulation is delivered via electrodes 40 and 42 of lead 18, a sensing vector may include any of electrodes 40, 42, and 62 from lead 18, or any of electrodes 44, 46, 48, 50, 58, 64, and 66 associated with other leads or with IMD housing 60. Accordingly, in some examples, delivery of electrical stimulation and the sensing of an evoked signal may utilize electrodes from a single implantable lead.

In other examples, delivery of stimulation and sensing of an evoked signal may be performed using electrodes on different leads 18, 20, 22. In other words, for an IMD comprising first and second implantable leads, the stimulation generator may be coupled to deliver the electrical stimulation via the first lead, and the sensing module may be coupled to receive the sensed evoked signals via the second implantable lead, and vice versa. In this manner, sensing integrity may be evaluated for a given vector based on evoked signals generated in response to stimulation delivered by that vector or evoked signals generated in response to stimulation delivered by other vectors. The first lead may the RV lead, and the second lead may be the LV lead, or vice versa. Alternatively, the sensing vector and stimulation vector may include the same electrodes on the same lead or leads. In general, the sensing integrity of a given vector can be evaluated with or without regard to the particular vector that produced the stimulation that caused an evoked cardiac potential.

As an illustration, electrical pacing stimulation may be delivered to RV 28 of heart 12, via electrodes 40 and 42 of lead 18, and the cardiac signal evoked by the electrical stimulation may be sensed by a unipolar sensing vector including elongated electrode 62 and electrode 58. IMD 16 may also deliver defibrillation and/cardioversion stimulation via elongated coil electrode 62. Accordingly, electrode 62 may be described as a high voltage or high energy coil electrode. Hence, in some aspects of this disclosure, sensing integrity may be evaluated based on evoked signals sensed by electrodes normally used for pacing, or for other functions such as cardioversion/defibrillation, but not necessarily used normally for sensing. Further, in this manner, sensing integrity may be evaluated while pacing pulses are also being delivered, including situations when IMD 16 may be delivering pacing therapy 100 percent or substantially 100 percent of the time to heart 12. In addition, in some cases, such a configuration may allow for delivery of stimulation and sensing of the signals evoked by the stimulation via electrodes contained on the same lead.

Although sensing may not typically be performed via coil electrodes 62, 64, 66, for example, it may be desirable to use such electrodes (or similar electrodes) to sense evoked signals for purposes of evaluating sensing integrity, in accordance with some aspects of this disclosure. In particular, evaluation of sensing integrity for vectors that include elongated electrodes 62, 64, 66 may facilitate, indirectly, an evaluation of lead integrity. In other words, to the extent sensing integrity can be used to infer lead integrity for purposes of reliable stimulation, it may be desirable to sensed evoked potentials along vectors that may not ordinarily be used for sensing. Hence, although coil electrodes 63, 64, 66 may ordinarily be used only for delivery high energy stimulation waveforms, such as cardioversion or defibrillation waveforms, such electrodes may be used as sense electrodes for sensing of evoked signals. In this manner, IMD 16 may sense at least some of the evoked signals using a sensing vector that includes at least one coil electrode or other high energy electrode used for delivery of high energy stimulation.

If can electrode 58 and coil electrode 62 are used to sense an evoked potential generated in response to stimulation delivered via electrodes 40, 42, for example, the sensed evoked potential may provide an indication of the functional and/or structural integrity of electrode 62, especially if the evoked potential sensed via electrode 62 is trended over time, e.g., by comparing evoked signal amplitudes to mean evoked signal amplitudes collected over time. As one illustration, if the amplitude of the evoked potential sensed via electrode 58 and electrode 62 falls substantially below a mean amplitude over time, the reduction in amplitude may indicate a possible lead-related condition associated with lead 18 and/or electrode 62. The possible lead-related condition for purposes of sensing integrity may, in turn, be used to infer lead integrity for purposes of stimulation delivered by lead 18 and/or electrode 62. Many other statistical measures of the evoked signal may be used to evaluate sensing integrity.

In some cases, IMD 16 or programmer 24 may be configured to evaluate oversensing via a sensing vector. For example, IMD 16 may be configured to analyze sense data provided by different sensing vectors, and cross-correlate them with one another or with marker channel information indicating when a pulse or other stimulation signal was delivered. If a sensing vector senses an evoked signal at a time when, in fact, other data show that the evoked signal should not have been detected, then IMD 16 or programmer 24 may determine that a potential lead-related condition or other sensing integrity condition may exist with respect to the vector.

IMD 16 may not be limited to sensing evoked cardiac signals via a single sensing vector. Rather, to evaluate sensing integrity on a more comprehensive basis, it may be desirable to sensed evoked cardiac signals via multiple vectors. In other words, to verify sensing integrity and, inferentially, lead integrity for multiple leads and electrode combinations, IMD 16 may be configured to obtain sensed evoked signal data from multiple vectors. Then, IMD 16 or programmer 24 may evaluate the individual vectors to determine whether a lead-related condition or other sensing integrity condition may exist.

In this manner, the reliability of multiple leads may be assessed contemporaneously, in addition to providing a means for verification of an evoked cardiac potential signal sensed by one or more leads. Hence, multiple sensing vectors may be used to sense the same evoked cardiac signal. Such an arrangement may allow the sensed cardiac signals sensed by each of the respective sensing vectors to be analyzed independently, as discussed above. Additionally, or alternatively, in some implementations, IMD 16 or programmer 24 may analyze the evoked signals obtained by multiple sense vectors with respect to one another. In particular, IMD 16 or programmer 24 may compare data from different sensing vectors to determine whether sensed events are consistent across the vectors and, therefore, reliable, or whether one or more vectors has produced data that may be unreliable and indicative of a sensing or lead integrity condition for the respective vector.

The analysis of the sensed cardiac signal may include comparing one or more properties exhibited by the sensed signal to the properties expected to be exhibited by a signal sensed by one or more reliable leads. If the comparison indicates one or more differences between the sensed cardiac signal and the signal expected from one or more reliable leads, the determination may be made based on the analysis that the one or more implantable leads are not reliable, e.g., such as the one or more of the implantable leads including the one or more electrodes used by the IMD to sense the evoked cardiac signals.

Conversely, if the comparison indicates that the sensed signal is consistent with the signal expected from one or more reliable leads, the determination may be made based on the analysis that the one or more implantable leads are reliable. In other examples, using the same or similar principles, the analysis of the sensed cardiac signal may include comparing one or more properties exhibited by the sensed signal to the properties expected to be exhibited by a signal sensed by one or more unreliable leads, instead of a reliable lead, as described above.

Analysis of the sensed evoked cardiac signal may include evaluating any of a variety of characteristics of the signal, including characteristics generated over time, such as trend data. As examples, the sensed evoked signal may be analyzed to track, over time, mean amplitude, mean time between a pace and a resulting evoked signal, percent change in amplitude over time, deviation of the evoked signal from one or more absolute thresholds or mean thresholds, counts of number of times the evoked signal deviated from absolute or mean thresholds, correlation of the evoked signal with a waveform template on a beat-to-beat or periodic basis, deviation from time or frequency domain norms for the waveform, deviation of the area under the evoked signal curve, slopes, inflection points or the like from a reference, or any combination of these or other statistical process control metrics relating to the evoked signal.

Amplitude may refer to a peak amplitude of a signal, such as a peak amplitude of an R wave associated with an evoked signal. The evoked signal may be filtered and rectified to facilitate analysis. The amplitude may be a voltage or current amplitude, but typically will be a voltage amplitude. Sensed signals may be compared, for example, to absolute or mean minimum amplitude thresholds, absolute or mean maximum amplitude thresholds, or maximum percent change thresholds on a pace-by-pace, periodic or longer term basis. In some implementations, more complex morphological characteristics, such as slope, frequency, signal width or the like may be evaluated.

Once IMD 16 has collected sensed evoked signal data, IMD 16 or programmer 24 may trend the data to generate any of the above characteristics over time. Upon collection and trending of this data, sensing and lead integrity may be evaluated automatically by IMD 16 or programmer 24, e.g., by applying one or more algorithmic analysis techniques. Alternatively, a user such as a clinician may evaluate sensing and lead integrity by visual inspection of the trend data, e.g., in numeric or graphic form. For example, programmer 24 or another external device may present a trend of the sensed evoked potential data to a user via presentation of a graph over time on a display. In either case, the automated or physician-directed evaluation may be made based on analysis of the sensed evoked signal data obtained by IMD 16 during use of the IMD over time. Based on the evaluation, in the case of automated analysis, IMD 16, programmer 24 or another external device may generate an indication of sensing integrity.

As described above, evaluation of sensing integrity may be performed based on trend data generated over time. IMD 16, programmer 24 or another external device may compare a particular sensed evoked signal to the trend data to determine if the sensed evoked signal deviates from the trend data by more than a threshold amount. In other cases, a short-term trend may be compared to long-term trend data to determine whether there is a potential sensing integrity condition. Alternatively, IMD 16 or programmer 24 may analyze long-term trend data to identify substantial deviation from a prescribed threshold value, such as maximum amplitude, average amplitude, minimum amplitude, percent change of amplitude, e.g., from an average amplitude, or the like.

As an illustration, if the amplitude of a particular evoked signal, as sensed by a particular sense vector, exceeds a mean amplitude by more than a threshold amount, it may be determined that a possible sensing integrity condition exists for that sense vector. As a further illustration, if the amplitude of a sensed signal is below a mean amplitude by more than a respective threshold amount, it may be determined that a possible sensing integrity condition exists for that sense vector. As a further illustration, if a specified number of evoked signals fall above or below a mean amplitude value by pertinent threshold amounts such that the number of deviations indicate a statistically abnormal condition, it may be determined that a possible sensing integrity condition exists for that sense vector. The specified number may be fixed or programmable.

As another illustration, if short-term trend data for a period of days, weeks, or months indicates a mean amplitude that deviates substantially from a longer-term mean amplitude, it may be determined that a possible sensing integrity condition exists. In such cases, IMD 16, programmer 24, another external device, or a clinician may indicate a sensing integrity issue and infer a possible sense circuit condition or lead integrity condition for a lead carrying one or more electrodes associated with the sensing vector.

As another illustration, if the amplitude of a particular evoked signal is below an absolute minimum threshold value, it may be determined that a possible sensing integrity condition exists for that sense vector. As another illustration, if the amplitude is above an absolute maximum threshold value, it may be determined that a possible sensing integrity condition exists for that sense vector. If a fixed or programmable number of signals fall below the absolute minimum threshold value or above the absolute maximum threshold value, it may be determined that a possible sensing integrity condition exists.

As another illustration, the area under a curve created by a sensed signal may be compared to a template or baseline amount. The template comparison may be performed, e.g., using digital correlation analysis. In this case, deviation from the template may be assessed by comparing a percentage of correlation to a threshold. If the number of signals found to be inconsistent with the template or baseline amount is statistically significant, i.e., if the number of signals that deviate from the template or baseline exceeds a threshold number, it may be determined that a possible sensing integrity condition exists for that sense vector. Again, a sensing integrity condition may generally refer to a condition that may alter sensing operation relative to a normal or desired operation.

As another illustration, the measured time between the leading edge of a pace pulse and the peak of the sensed evoked signal may be compared to an expected time value, such as an absolute value or a mean time value for signals collected over time. If this amount of time is inconsistent with an expected, preestablished or trended amount, it may be determined that a possible sensing integrity condition exists for that sense vector.

As another illustration, signal waveforms, e.g., either raw or filtered waveforms, may be analyzed for one or more of time or frequency domain changes. If a number of anomalies between sensed and expected time domain or frequency domain characteristics is found to be statistically significant, i.e., by comparison to a threshold number, it may be determined that a possible sensing integrity condition exists for that sense vector.

In some examples, if it is determined that a possible sensing integrity condition exists for that sense vector, e.g., based on one or more of the techniques described in this disclosure, IMD 16 or programmer 24 may respond to the determination in a variety of ways. For example, IMD 16 or programmer 24 may immediately alert a patient and/or physician of the possible sensing integrity condition. As another example, IMD 16 or programmer 24 may perform a higher resolution algorithm to confirm or isolate the possible sensing integrity condition. A so-called higher resolution algorithm may result in an increased signal sampling rate, or an increase in the amount or type of signal information that is analyzed. As another example, one or more different sensing vectors may be utilized to sense evoked signals to confirm the sensing integrity condition. In some examples, one or more of these steps may only be taken after the analysis of the evoked signal has indicated a possible sensing integrity condition over a programmable period of time or over a programmable number of signals, while in other examples, a single indication will trigger such a response.

As described above, sensed evoked signal data may be analyzed based on various trend data, such as mean amplitude values. Alternatively, one or more instantaneous or trended signal characteristics may be compared to preprogrammed values. The preprogrammed values may be used initially as a starting point until sufficient data are collected to develop trend data. Alternatively, preprogrammed values such as threshold amplitude values may be used continuously for comparison to individual sensed evoked signal samples or shorter-term trend data relating to multiple sensed evoked signals. The preprogrammed values, if used, may be patient-specific and may be selected by a clinician. In either case, if a given sensed evoked signal or a trended sensed evoked signal deviates from such a value, whether preprogrammed or trended over time, the evaluation may indicate a possible sensing integrity condition.

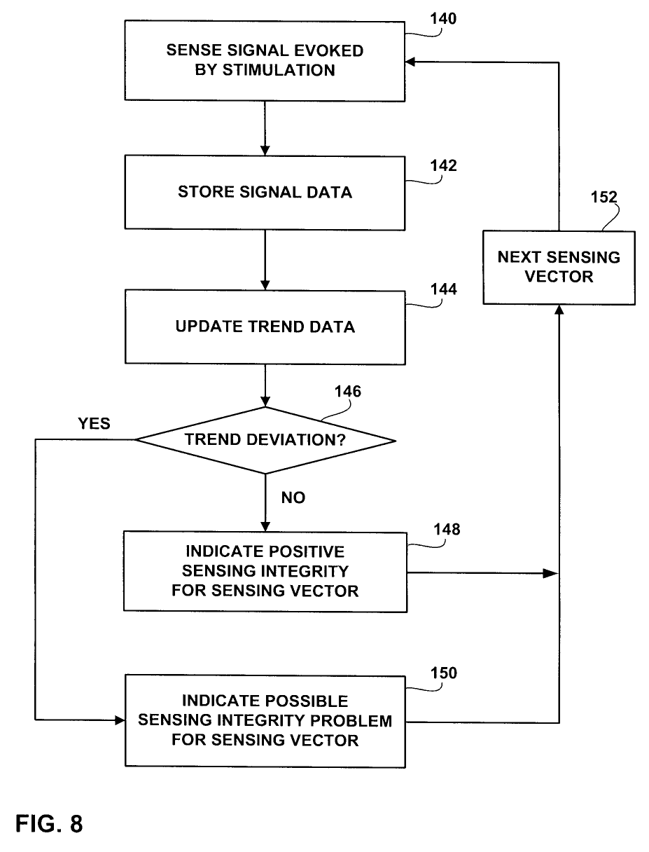

FIG. 8 is a flowchart that illustrates an example technique for evaluating sensing integrity. The technique shown in FIG. 8 may be utilized by system 10 to determine sensing integrity for one or more sensing vectors. The technique may be used, in turn, to infer the reliability of one or more implantable leads 18, 20, and 22 associated with such sensing vectors. If sensing integrity is altered, a lead associated with the sensing vector may suffer from one or more lead-related conditions, such as open or short circuits, low or high impedances, or the like. The lead-related conditions may be due to damage of the lead.

In the example of FIG. 8, IMD 16 senses a cardiac potential signal evoked by the delivery of electrical stimulation to tissue of heart 12 of patient 14 (140) via a sensing vector comprising a selected set of electrodes. IMD 16 may store data relating to the sensed evoked signal (142) and update applicable trend data (144) based on the sensed evoked signal. For example, if trend data includes a mean amplitude, IMD 16 may update the running mean amplitude based on the amplitude of the sensed evoked signal.

In some implementations, IMD 16 may store raw signal data, store processed data, or store the processed data or trend data (144) without raw signal data. The raw signal data may include sufficient data to permit reconstruction of one or more characteristics of the signal. Alternatively, the characteristics included in the raw signal data may be parametric characteristics, e.g., based on information extracted by a DSP in sensing module 86 as previously described, such as signal amplitude, frequency, or the like. In either case, evoked signal data may be stored with absolute or relative time stamps to permit trending of the data over time. The trend data may be updated within IMD 16. In other implementations, trending of data and associated updating of the trend data may be performed remotely, e.g., in a programmer 24 of other external device.

IMD 16, programmer 24 or another external device may analyze the trend data and/or the sensed evoked signal data relative to the trend data to determine whether there is a deviation from the trend (146). Alternatively, a clinician or other user may inspect the trend data, e.g., visually. The trend deviation may be, for example, a substantial deviation of the sensed evoked signal from an established trend. For example, it may be determined whether the sensed evoked signal has an amplitude that is greater or less than a mean amplitude by more than a threshold amount. The same or different threshold amounts may be used for underage and overage of the amplitude relative to the mean amplitude.

Alternatively, or additionally, it may be determined whether a short-term trend, such as a mean amplitude over shorter period of time such as a specified number of days, deviates significantly from a longer-term trend, such as an average amplitude over a period of weeks or months. The short-term trend amplitude may deviate from the longer-term trend amplitude if it is over or under the long-term mean amplitude by more than a threshold amount. In either case, a deviation from the trend data may be identified to indicate a possible loss in sensing integrity.

In other examples, the long-term trend may be analyzed relative to applicable predetermined threshold values, such that loss in sensing integrity may be indicated if the long-term mean amplitude deviates from a predetermined threshold value by a specified amount. Alternatively, loss of sensing integrity may be indicated if the long-term mean amplitude changes by more than a predetermined percentage of the mean amplitude in a predetermined unit of time. As an illustration, if the mean amplitude changes (e.g., upward or downward) by more than X percent over a period of N days, IMD 16, programmer 24 or another device may indicate a possible loss of sensing integrity.

If there is no substantial deviation from the trend (146), e.g., the amplitude of the evoked signal is within the threshold amount of the mean amplitude, the evaluation may indicate a positive sensing integrity for the sensing vector by which the sensed signal and trend data were obtained (148). In some cases, the positive indication of sensing integrity may permit an inference of positive lead integrity for the lead or electrodes associated with the pertinent sensing vector. If there is a deviation from the trend (146), the evaluation may indicate a negative sensing integrity for the sensing vector by which the sensed signal and trend data were obtained (150). In some cases, the negative indication of sensing integrity may permit an inference of negative lead integrity for the lead or electrodes associated with the pertinent sensing vector.

Upon completing the evaluation of sensing integrity for a given sensing vector, e.g., based on comparison of a sensed evoked signal or short-term trend to a longer-term trend, the process outlined in FIG. 8 may proceed to evaluate sensing integrity for another sensing vector (152), such as the next sensing vector in a predefined progression of sensing vectors to be evaluated for sensing integrity. The process may continue for multiple sensing vectors to permit evaluation of sensing integrity across multiple leads and electrodes. Once all desired sensing vectors are evaluated for sensing integrity, the process may continue for the next sensed evoked signal data or collection of trend data.

In some implementations, evaluation of sensing integrity may be performed dynamically for each newly sensed evoked signal obtained by IMD 16. In this case, to evaluate sensing integrity as each new signal is sensed, sensing integrity may be evaluated within IMD 16, or possibly by streaming sensed evoked signal data to a programmer 24 or other external device. As an alternative, evaluation of sensing integrity may be performed periodically for a set of trend data collected over time. For example, IMD 16 may periodically evaluate the trend data on a regular basis, or on-demand in response to an internally generated event or a command received from an external programmer 24. As another example, an external programmer 24 or another device may interrogate IMD 16 to retrieve a set of sensed data or collected trend data for evaluation by the programmer or device. In the case of sensed data, the programmer 24 may process the data to produce trend data. The programmer 24 or other device may automatically analyze the collected trend data to evaluate sensing integrity or present a visual representation of the data to a user, e.g., for visual inspection.

Whether sensing integrity is evaluated on a signal-by-signal basis or by reference to a collection of trend data, IMD 16 may be configured to obtain the sensed evoked signals for different sensing vectors at a regular or irregular sampling rate. Evoked signals may be sensed substantially simultaneously using different sensing vectors. Alternatively, each sensing vector may have a dedicated sampling time that is independent of other sensing vectors. In each case, the rate at which evoked signals are sensed may be fixed or variable and may be a high or low rate. As an illustration, IMD 16 may be configured to obtain sensed evoked signals several times per week, day, hour, or minute, e.g., subject to clinician preferences or operational capabilities of the IMD.

When a potential trend deviation is detected, IMD 16 may increase the sampling rate periodically so that additional data for a possible loss in sensing integrity can be quickly collected for investigation. In general, an evaluation of sensing integrity and, in turn, the reliability of one or more leads, may be performed on a continuous or periodic basis. For example, sensed cardiac signal information stored by IMD 16 may be uploaded to an external device in response to an event, on-demand, periodically, e.g., on a daily, weekly or monthly basis, or at a patient clinic visit.

In some implementations, the trend data may be specifically generated and tracked for respective sensing vectors, or respective implantable leads, instead of using a universal set of trend data for all sensing vectors. In this manner, analysis may take into account any inconsistencies between sensing vectors and/or implantable leads that may indicate unreliability when compared to a universal baseline, but are in fact related to inherent differences between respective sensing vectors rather than a result of one or more unreliable leads. In this manner, the reliability of one or more implantable electrodes may be more accurately determined.

In some situations, it may be determined that one or more leads 18, 20, 22 are not reliable based on the evoked cardiac signals sensed by IMD 16. However, the identification of the specific lead(s) that are not reliable may not be readily obtainable, e.g., based on the sensing vector utilized to sense the evoked cardiac signals. In particular, the sensing vector may include electrodes from two different leads, or electrodes from one lead and the IMD housing. Furthermore, in some situations it may be desirable to confirm a reliability determination with respect to one or more leads based on the analysis of the sensed cardiac signals. In such situations, IMD 16 may carry out one or more procedures according to sensing protocols designed to verify a reliability determination and/or identify an individual unreliable lead that is part of a plurality of leads that have been determined not reliable. For example, when a possible loss of sensing integrity is detected for a sensing vector that involves electrodes from different leads, it may be desirable for IMD 16 to evaluate other sensing vectors that isolate the electrodes in conjunction with electrodes on the same leads.

The analysis and/or determination of the reliability of one or more implantable leads based on sensed cardiac signals evoked from electrical stimulation as described herein may be carried out by a variety of devices, as described above. In some examples, the analysis and evaluation of sensing integrity, and reliability of an implantable lead, may be carried out entirely by IMD 16. In other examples, the analysis and evaluation of sensing integrity may be carried out entirely by an external device, e.g., programmer 24 shown in FIG. 1. In such examples, IMD 16 may simply sense cardiac signals evoked by the delivery of stimulation and store raw data or parametric data associated with the signals in memory 82 for later retrieval via telemetry module 88 by the external device for analysis of the information. In still other cases, the analysis and evaluation of sensing integrity based on sensed evoked cardiac signals may be carried out in part by IMD 16 and in part by an external device, e.g., programmer 24. For example, IMD 16 may analyze the sensed cardiac signals, generate trend data, and make the determination that further analysis should be performed by programmer 24. In such cases, IMD 16 may generate an indication to alert programmer 24 or patient 14 that it may be advisable to perform further analysis to determine the reliability of one or more leads of IMD 16.

Examples of the external devices that may analyze and/or determine the reliability of one or more leads 18, 20, and 22 of IMD 16 are not limited to programmer 24. In some instances, an external device such as a server or other computing appliance on a network coupled to programmer 24 and/or IMD 16 may perform at least a portion of the analysis and evaluation of sensing integrity. Such a device may receive information from IMD 16 and/or programmer 24 and simply present the data to a clinician or other user situated at a remote viewing terminal. In other cases, such a device may process the data to produce trending data, analyze the data, and/or evaluate sensing integrity based on the analysis. For example, a device may analyze sense data, produce a trending report, and send the trending report to a clinician, e.g., as a web page or other document for viewing via a web browser or other viewing application.

IMD 16, programmer 24 or another device may be configured to generate a positive or negative indication of sensing integrity. In some cases, the indication may quantify a degree of sensing integrity, e.g., based on an amount of trend deviation or deviation of sensed evoked signals data relative to some other standard or threshold A determination of reliability of one or more leads may include indicating the reliability based on what has been determined for the one or more implantable leads. In some examples, if IMD 16 determines that a particular sensing vector or lead is not reliable based on the analysis of the sensed evoked cardiac signals, IMD 16 may alert a patient or physician (e.g., by audible or tactile alerts and/or wireless telemetry) of the sensing integrity determination, or simply record the determination, e.g., in memory 82, to be accessed and reviewed by a clinician at a later time.

The nature of the indication may depend on an estimated degree of unreliability determined for the one or more leads. For example, the analysis of sensed evoked cardiac signals may be consistent with an intermittent lead integrity issue that does not require attention immediately. Instead, it may be advisable that a patient schedule an appointment with a physician in the near future to investigate the issue. In such a case, the indication may include a message, notification or alert delivered by the IMD 16 to the patient or caregiver via a network, e.g., upon wireless telemetry between the IMD 16 to a network access point. The message may indicate that an appointment is advisable.

As another example, the analysis of sensed cardiac signals may be consistent with a lead integrity issue that requires attention immediately. In such as case, the indication may include an emergency alert to the patient and/or caregiver indicating a need for immediate attention. For example, a patient may receive a type of sensory stimulation, e.g., an audible or tactile indication via IMD 16. A tactile indication may be a vibrational indication. In any event, such an indication may be commensurate with the perceived importance of the reliability determination. However, an indication is not limited to situations in which a determination of unreliability is made. Instead, is some examples, an indication that one or leads are reliable may accompany a positive sensing integrity determination based on the sensed evoked cardiac signal. In general, different types (e.g., in terms of patterns, amplitudes, or the like) of audible or tactile indications may be used to indicate different levels of urgency.

Although all or portions of the analysis and evaluation of sensing integrity may be performed by one or more devices as described above, in some examples, a user may analyze and/or determine the reliability of one or more implantable leads based on the analysis of sensed evoked cardiac signals. In some cases, this may include a visual inspection of a representation of the sensed evoked cardiac signals by a user, e.g., a physician, clinician, technician or other caregiver. For example, a user may retrieve sensed cardiac signal information stored in memory 82 of IMD 16 via programmer 24. The signal information may be raw signal data, trend data or processed data. Programmer 24 may display a graphical representation of the retrieved information via user interface 104. The graphical representation may include a representation of values, such as mean amplitude, over a period of time, e.g., in a value versus time plot or other arrangement. Based on the displayed representation, the user may be able to analyze the sensed cardiac signal data and evaluate sensing integrity based on the analysis of the sensed data.

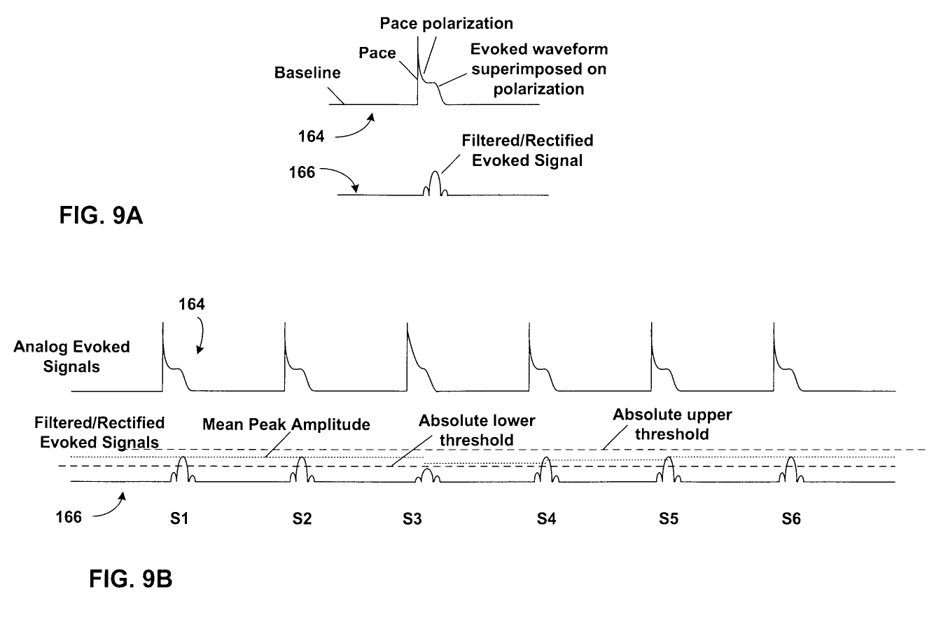

FIG. 9A is a graphical representation of an example single sensed evoked cardiac signal waveform. In particular, waveform 164 is representative of an analog signal that may be received by sensing module 86 of IMD 16 according to an evoked signal sensed by one or more sensing vectors on leads 18, 20, or 22 following delivery of a pacing pulse. Waveform 64 shows a signal baseline, followed by a pace, i.e., pacing pulse. The pacing pulse produces a pace polarization in the cardiac tissue, as shown in waveform 164. Waveform 164 further includes an evoked waveform that is superimposed on the polarization. Waveform 166 is representative of a digital waveform 164 following filtering and rectification, e.g., with a DSP. As previously described, in some examples, such a digital signal may be generated by a DSP, e.g., in sensing module 86 of IMD 16, to extract information useful in the evaluation of the signal for purposes of evaluating sensing integrity, and the information may be stored via memory 82 of IMD 16.

The stored information may be related to any of a variety of waveform characteristics. For example, IMD 16 or another device may extract characteristics such as signal amplitude, signal frequency, area under the signal waveform, signal slope, signal inflection points, or the like. IMD 16 or another device may analyze the signal in a variety of ways. For example, one or more characteristics may be compared to pertinent thresholds. The thresholds may be absolute thresholds or trend-based thresholds. As another example, the characteristics may be compared to applicable waveform templates, e.g., by a digital correlation process. The evoked signals may be analyzed on a beat-to-beat, continuous or periodic basis.

FIG. 9B is a graphical representation of an example series of sensed evoked cardiac signal waveforms 164 and the corresponding series of converted digital signal waveforms 166 over a period of time. The converted digital signal waveforms 166 in FIG. 9B are filtered and rectified versions of the actual waveform 164 sensed by IMD 16, e.g., as described above with reference to FIG. 9A. In the example of FIG. 9B, it is assumed that IMD 16 or another device such as programmer 24 is configured to sense an evoked signal and convert the signal to a form that can be processed to readily identify a peak amplitude of the signal.

IMD 16, programmer 24 or another device may analyze the signal waveform in any of a variety of ways. Some example signal analysis methods are described in this disclosure for purposes of illustration and without limitation to the variety of techniques that may be used. As one example, wide band raw or filtered signal data may be collected, either continuous or in one beat snippets, and stored for processing, either within IMD 16, programmer 24, or elsewhere. In one implementation, the collected wide band raw or filtered signal data may be compared to a digital template, periodically or beat to beat, and the degree to which the signal for each subsequent beat matches the template may be determined. If the degree of matching is within a predetermined range, sensing integrity may be determined to be positive. If the degree of matching is outside the predetermined range, then a notification of loss of sensing integrity may be generated, further analysis of confirmation may be performed, or both.

As a further example, the signal amplitude may be compared to one or more absolute thresholds, one or more running averages or mean values or other threshold values. If the signal amplitude is above or below an applicable absolute threshold or above or below a running average or mean value by a predetermined amount, IMD 16, programmer 24 or another device may generate an indication of possible loss of sensing integrity, e.g., as a flag. The predetermined amounts may be fixed, adaptive and/or programmable. In addition, events that are statistically abnormal, such as too many consecutive beats above the average or too many consecutive beats below the average may be flagged and result in an indication of possible loss of sensing integrity.

As another example, an area under the curve of the raw or filtered waveform for each beat may be compare to the area under the curve for a template or baseline waveform. Events indicating substantial deviation of the area under the curve from the template or baseline area by more than a threshold amount may be flagged, and provide an indication of possible loss of sensing integrity.

In addition, the time from the leading edge of a pacing pulse to the peak of the evoked signal may be measured, and compared to an expected value, so that times that are longer or shorter than the expected value may be flagged as being indicative of possible loss of sensing integrity. The expected value may be an absolute lower value, an absolute upper value, a mea value or a combination of such values. As a further example, the raw or filtered waveform may be analyzed in the time domain or frequency domain to identify other signal anomalies.

In any case, one events are flagged, IMD 16, programmer 24 or another device may generate an immediate alert or other notification indicating loss of sensing integrity, perform a higher resolution algorithm to confirm the loss of sensing integrity, or confirm the loss of sensing integrity with signals obtained via other sensing vectors. In some implementations, a programmable number of accumulated flagged events may be required before generating an alert or other notification of loss of sensing integrity.

As discussed above, a variety of analytical techniques may be used to evaluate loss of sensing integrity based on sensing of evoked signals. Some of such techniques are described in more detail below.

IMD 16, programmer 24 or another device may trend the peak amplitude data over time to produce a mean peak amplitude. The mean amplitude may refer to a mean peak amplitude of the processed signal relative to a reference level. The mean peak amplitude may be updated over time as additional evoked signals are newly sensed for a pertinent sensing vector of IMD 16. Further, FIG. 9B also illustrates an absolute lower threshold amplitude level and an absolute upper amplitude threshold which define a amplitude values to which a single evoked signal amplitude may be compared to as previously described.

For example, to evaluate sensing integrity, the peak amplitude of an individual evoked signal may be compared to the absolute lower threshold value and the absolute upper threshold value. If the peak amplitude of the evoked signal falls below the absolute lower threshold value, or rises above the absolute upper threshold value, then IMD 16, programmer 24 or another device may indicate that there may be a sensing integrity condition.

Alternatively, instead of comparing a single amplitude to the absolute threshold values, the IMD 16 or programmer 24 may compare a mean peak amplitude of the signals over a period of time to the upper and low threshold values. For example, if the mean peak amplitude is greater than the absolute upper threshold or lower than the absolute lower threshold, a sensing integrity condition may be indicated. The mean peak amplitude may be a mean peak amplitude maintained over a long period of time, or a shorter period of time.

Although absolute upper and lower thresholds are shown in FIG. 9B, in some embodiments, only one of the threshold values may be used. For example, comparison of the signal amplitude to an absolute lower threshold value may be sufficient in some implementations.

Alternatively, or additionally, IMD 16, programmer 24 or another device may monitor the peak signal amplitude relative to a mean peak amplitude. In this case, rather than comparing the peak signal amplitude to absolute thresholds, the peak signal amplitude may be analyzed to determine whether it deviates from the mean peak amplitude by more than a particular amount. In some implementations, the amount may be a fixed or adjustable amount. The amount may be a percentage of the mean peak amplitude, and may be the same or different for peak signal amplitudes above the mean peak amplitude and peak signal amplitudes below the mean peak amplitude. For example, IMD 16, programmer or another device may indicate a potential sensing integrity condition if the peak signal amplitude exceeds the mean peak amplitude by more than X percent or falls below the mean peak amplitude by more than Y percent, where X and Y are the same or different. The mean peak amplitude may be trended over time and calculated as a mean of the peak amplitudes of the evoked response signals obtained for analysis over time.

For example, IMD 16 or programmer 24 may analyze an overall long-term trend to determine if it has deviated from a preprogrammed threshold, deviated by a predefined percentage from a previously determined amplitude or mean amplitude, or otherwise indicates a trend deviation that may be predictive or indicative of a loss of sensing integrity. In some implementations, individual signals, short-term trends, and long-term trends may be analyzed in conjunction with one another, each according to its own threshold or thresholds, such that deviation in any of them by pertinent amounts may trigger an indication of a possible sensing integrity condition. A wide variety of different approaches may be applied. Accordingly, the techniques described in this disclosure are provided for purposes of illustration, and without limitation to the general techniques broadly described in this disclosure. In each case, evoked signals are used to evaluate sensing integrity.

As an illustration, in FIG. 9B, IMD 16 has collected multiples samples over time. In the simplified example of FIG. 9, IMD 16 has collected signal samples S1-S6 at different times and developed a mean peak amplitude as a form of trend data that may be updated as each new sample is collected. Again, data can be collected multiple times per minute, hour, day, or week, subject to various design considerations and clinician preferences. The signal samples shown in FIG. 9B are limited to a small number only for purposes of ease of illustration. If IMD 16, programmer 24 or another device is configured to identify evoked signals having amplitudes that fall below the absolute lower threshold amplitude, then it may identify sample S3 as indicating a possible loss of sensing integrity. Alternatively, IMD 16, programmer 24 or another device may indicate a possible loss of sensing integrity if an evoked signal has an amplitude that is above an absolute upper threshold amplitude, or if the amplitude deviates from the mean amplitude by more than a predetermined amount, such as a predetermined percentage.

As illustrations, IMD 16 or programmer 24 may be configured to detect deviation of the peak amplitude of a single sample from the mean peak amplitude by more than a first threshold amount, deviation of a mean peak amplitude for a fixed number of consecutive samples (a shorter term trend) from the longer-term mean peak amplitude by more than a second threshold amount, and/or deviation of the overall, longer-term mean peak amplitude from a predetermined level by more than a third threshold amount or percentage. Again, the amplitudes may be voltage amplitudes, although the techniques may be configured to analyze current amplitudes or other signal characteristics. Moreover, with a DSP, amplitudes may be expressed in terms of digital values.

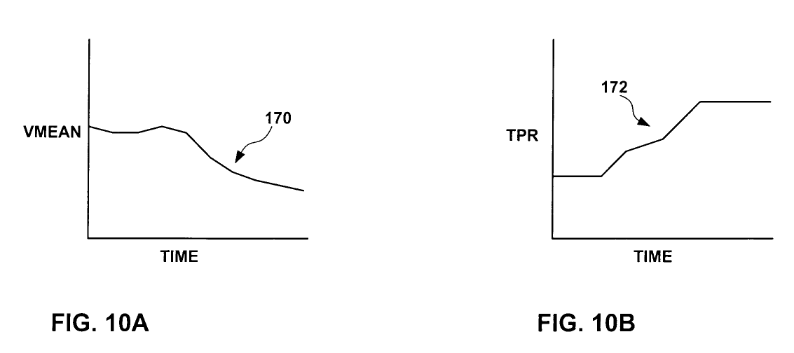

In some aspects, programmer 24 or another device may automatically detect a deviation as shown in FIG. 9B using automated signal or data analysis techniques, or present a graph similar to that shown in FIG. 9B to a clinician via a display or printout for visual inspection. A graph presented to a clinician, in some implementations, may omit representations of waveforms and may simply convey trend data, such as a plot showing changes in mean amplitude or other characteristics over a period of time, such as days, weeks or months, e.g., as shown in FIGS. 10A and 10B, discussed below. Accordingly, the waveforms are shown in FIG. 9 for purposes of illustration.

FIGS. 10A and 10B are graphical representations of trend data produced for sensed evoked cardiac signal waveforms over time. The example of FIG. 10A shows trend data representing a mean amplitude of sensed evoked signals over a period of time. The example of FIG. 10B shows trend data showing a mean time between application of a pacing pulse and sensing of an evoked signal over time. The time may be determined by the time that stimulation is applied versus the time that a peak amplitude of the evoked signal is detected. Changes in the time between application of a pacing pulse and sensing of an evoked response potential in response to such a pacing pulse may provide an indication of sensing integrity. In particular, the trend data may indicate a change in a time between delivery of the electrical stimulation and sensing of the evoked signals. The change in time indicated by the trend data may be analyzed to evaluate sensing integrity. If the change in time, in terms of either a shortening or lengthening, exceeds an applicable threshold amount of change, IMD 16, programmer 24 or another device may indicate a possible sensing integrity condition.

IMD 16 or programmer 24 may automatically analyze trend data as shown in FIGS. 10A and 10B to evaluate sensing integrity and generate an indication of sensing integrity. Alternatively, a clinician may view graphical information similar to that of FIGS. 10A and 10B and, by visual inspection, evaluate sensing integrity. In FIGS. 10A and 10B, reference numerals 170 and 172, respectively, indicate substantial changes in trend data that may be indicative of possible loss of sensing integrity. Upon automatically detecting such changes, or visually observing the changes in displayed or printed graph, an indication of loss of sensing integrity may be generated. In response, a clinician may take appropriate action to address the sensing integrity issue, and any lead-related conditions or other conditions that may be associated with the loss of sensing integrity.

Examples of this disclosure describe storing, in memory 82 of IMD 16, sensed evoked cardiac signal information that may be analyzed to determine sensing integrity and the reliability of one or more leads. However, memory 82 may store additional information that may be helpful or necessary in analyzing sensing integrity of IMD 16. For example, IMD 16 may also store absolute or relative times that an evoked cardiac signal was sensed by IMD 16 following a pacing pulse, e.g., as described above with reference to FIG. 10B.

As another example, IMD 16 may also store the specific sensing vector utilized to sense a respective signal. As another example, IMD 16 may also store information concerning one or more heart timing parameters determined by IMD 16, such as, e.g., A to V timing parameters, V to V timing parameters, R-R intervals, or the like, that may be coincident with sensed evoked signals. In this manner, additional information may be retrieved from memory 82 of IMD 16 to supplement the analysis of evoked cardiac signal sensed by IMD 16, and determination of sensing integrity and the reliability of one or more leads 18, 20, and 22 of IMD 16 based on the analysis.

In addition to analyzing sensing integrity and lead integrity for purposes of electrical sensing and stimulation via sense or stimulation electrodes provided on a lead, sensing integrity also may indicate integrity of other sensing features to be inferred. For example, in addition to indicating indicate reliability of sense electrodes, lead conductors, sense electronics, sensing integrity may also indicate reliability of non-electrode sensors, such as oxygen sensors, pressure sensors, accelerometers, ultrasound sensors, or the like, which also may rely on conductors within the lead, or sense electronics within the lead of the IMD housing. Accordingly, sensing integrity may indicate integrity of a wide range of sensing and stimulation features associated with an IMD.

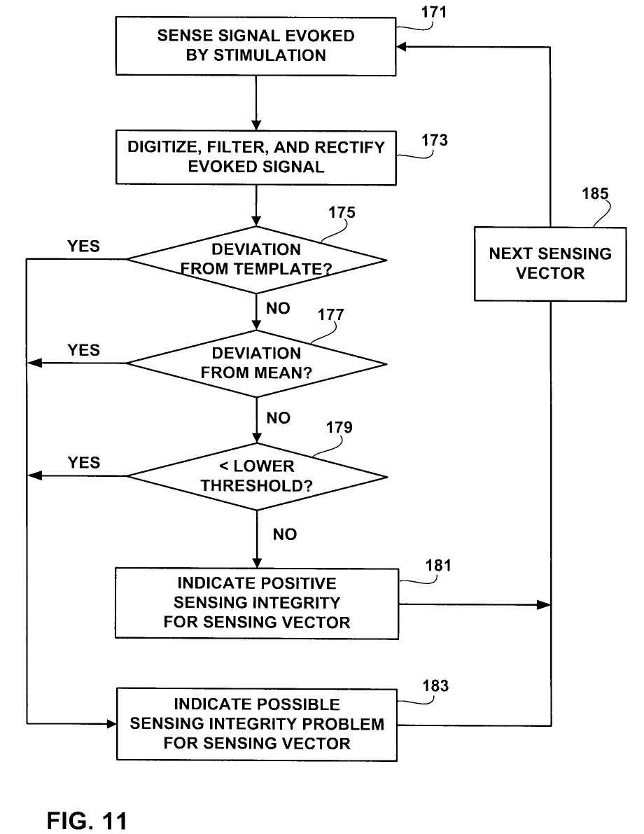

FIG. 11 is a flow diagram illustrating an example technique for evaluating sensing integrity using various statistical measures. As shown in FIG. 11, IMD 16 senses a signal evoked by stimulation on a particular sensing vector (171). IMD 16 may digitize, filter and rectify the evoked signal (173), and store the data for analysis within IMD 16, programmer 24 or another external device. The signal or signal data then may be analyzed to determine whether it deviates by more than a specified amount from a previously generated signal template (175). If the signal matches the template by less than a percentage amount, a possible sensing integrity condition may be indicated for the sensing vector (183).

If there is sufficient matching and, therefore, an acceptable amount of deviation from the template (175), the signal may be analyzed to determine whether it deviates from a mean (177), such as a mean peak amplitude developed over time. If the peak amplitude of the signal deviates from the mean peak amplitude (177) by more than a specified amount of deviation, e.g., a percentage, then a possible sensing integrity condition may be indicated (183).

If the signal does not substantially deviate from the mean (177), the signal may be analyzed to determine whether its amplitude is below an absolute lower threshold value (179). In some implementations, the signal may be, additionally or alternatively, compared to an absolute upper threshold value or other values. If the signal is not below the threshold value, a positive sensing integrity may be indicated for the sensing vector (181). If the signal is below the threshold value (179), however, a possible sensing integrity condition may be indicated (183).

In either case, the process may then continue to consider sensing integrity for additional sensing vectors (185). If a possible sensing integrity condition is indicated, IMD 16, programmer 24 or another device may generate an alert or other notification. Alternatively, additional processing, such as a higher resolution algorithm involving more measurements, more vectors, increased sampling rate, or the like, may be activated. Then, if the higher resolution algorithm indicates a possible sensing integrity condition, an alert or notification may be generated.

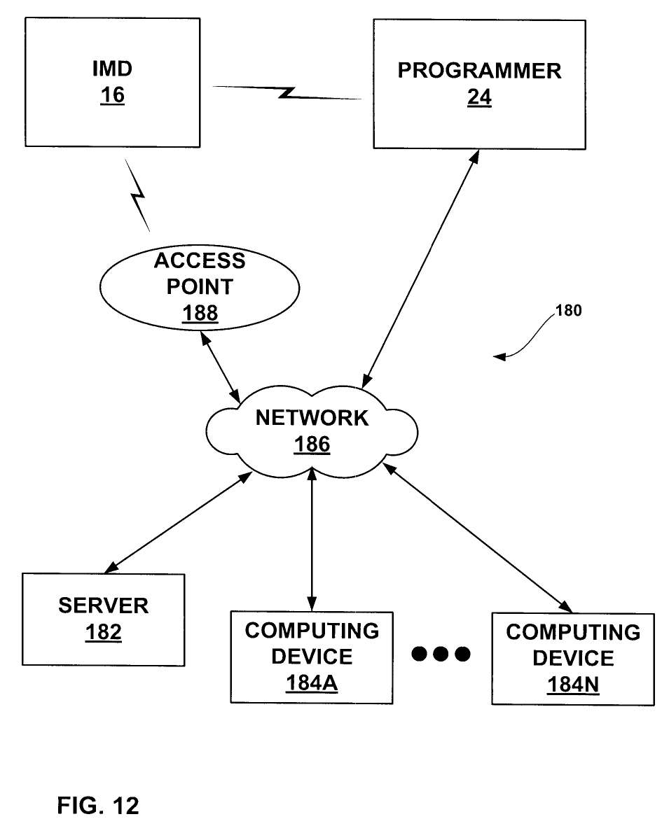

FIG. 12 is a block diagram illustrating an example system 180 that includes an external device, such as a server 182, and one or more computing devices 184A-184N, that are coupled to the IMD 16 and programmer 24 shown in FIG. 1 via a network 186. In this example, IMD 16 may use its telemetry module 88 to communicate with programmer 24 via a first wireless connection, and to communication with an access point 188 via a second wireless connection. In the example of FIG. 12, access point 188, programmer 24, server 182, and computing devices 184A-184N are interconnected, and able to communicate with each other, through network 186. In some cases, one or more of access point 188, programmer 24, server 182, and computing devices 184A-184N may be coupled to network 186 through one or more wireless connections. IMD 16, programmer 24, server 182, and computing devices 184A-184N may each comprise one or more processors, such as one or more microprocessors, DSPs, ASICs, FPGAs, programmable logic circuitry, or the like, that may perform various functions and operations, such as those described herein.

Access point 188 may comprise a device, such as a home monitoring device, that connects to network 186 via any of a variety of connections, such as telephone dial-up, digital subscriber line (DSL), or cable modem connections. In other embodiments, access point 188 may be coupled to network 130 through different forms of connections, including wired or wireless connections.

During operation, IMD 16 may collect and store various forms of evoked signal data. For example, as described previously, IMD 16 may collect sensed evoked signal data for sensing vectors including electrodes associated with one or more of leads 18, 20, and 22. In some cases, IMD 16 may directly analyze the collected data to evaluate sensing integrity and generate any corresponding reports or alerts with respect to sensing integrity. In other cases, however, IMD 16 may send stored data relating to sensed evoked signals to programmer 24 and/or server 182, either wirelessly or via access point 188 and network 186, for remote processing and analysis. For example, IMD 16 may sense, process, trend and evaluate the sensed evoked signals. Alternatively, processing, trending and evaluation functions may be distributed to other devices such as programmer 24 or server 182, which are coupled to network 186.

In some cases, IMD 16, programmer 24 or server may process sensing integrity data for one or more sensing vectors and leads into a displayable sensing integrity report, which may be displayed via programmer 24 or one of computing devices 184A-184N. The sensing integrity report may contain trend data for evaluation by a clinician, e.g., by visual inspect of graphic data. In some cases, the sensing integrity report may include an indication of positive or negative sensing integrity, or a quantification of a degree of sensing integrity, based on analysis and evaluation performed automatically by IMD 16, programmer 24 or server 182. A clinician or other trained professional may review and/or annotate the lead integrity report, and possibly identify any lead-related conditions.

In some cases, server 182 may be configured to provide a secure storage site for archival of sensing integrity information that has been collected from IMD 16 and/or programmer 24. Network 186 may comprise a local area network, wide area network, or global network, such as the Internet. In some cases, programmer 24 or server 182 may assemble sensing integrity information in web pages or other documents for viewing by trained professionals, such as clinicians, via viewing terminals associated with computing devices 184A-184N. System 180 may be implemented, in some aspects, with general network technology and functionality similar to that provided by the Medtronic CareLink® Network developed by Medtronic, Inc., of Minneapolis, Minn.

Although some examples of the disclosure may involve the sensing of evoked cardiac signals to monitor sensing integrity and reliability of one or more implantable leads in applications in which intrinsic cardiac signals are not sufficiently available, e.g., in CRT applications, the disclosure is not limited to such applications. Instead, the techniques described in this disclosure may be used for applications in which intrinsic cardiac signals are sufficiently available to monitor for lead related conditions. In some examples, evoked cardiac signals may be used to monitor for sensing integrity and lead related conditions in addition to intrinsic cardiac signals to provide corroborating determinations of sensing integrity and lead related conditions.

Furthermore, although the disclosure is described with respect to cardiac stimulation therapy, such techniques may be applicable to IMDs that convey other therapies in which lead integrity is important, such as, e.g., spinal cord stimulation, deep brain stimulation, pelvic floor stimulation, gastric stimulation, occipital stimulation, functional electrical stimulation, and the like. For some therapies, stimulation may likewise produce a depolarization signal or other signal that is evoked by stimulation tissue in response to the stimulation. In such therapies, the techniques described in this disclosure may be applied to evaluate sensing integrity and, in turn, detect possible lead-related conditions.

Also, in some aspects, techniques for evaluating sensing integrity, as described in this disclosure, may be applied to IMDs that are generally dedicated to sensing or monitoring and do not include stimulation or other therapy components. For example, an implantable monitoring device may be implanted in conjunction with an implantable stimulation device, and be configured to evaluate sensing integrity of leads or electrodes associated with the implantable monitoring device based on sensed signals evoked by delivery of stimulation by the implantable stimulation device.

The techniques described in this disclosure, including those attributed to image IMD 16, programmer 24, or various constituent components, may be implemented, at least in part, in hardware, software, firmware or any combination thereof. For example, various aspects of the techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable data storage medium such as random access memory (RAM), read-only memory (ROM), non-volatile random access memory (NVRAM), electrically erasable programmable read-only memory (EEPROM), FLASH memory, magnetic data storage media, optical data storage media, or the like. The instructions may be executed to support one or more aspects of the functionality described in this disclosure.

Various embodiments of the disclosure have been described. These and other embodiments are within the scope of the following claims.

1. A method comprising:
obtaining one or more sensed signals evoked by tissue in response to electrical stimulation of the tissue by an implantable medical device; and
evaluating sensing integrity of the implantable medical device based on analysis of the sensed signals.

2. The method of claim 1, wherein the tissue is cardiac tissue of a heart, and the electrical stimulation comprises cardiac pacing stimulation.

3. The method of claim 2, further comprising delivering the electrical stimulation to the tissue via an implantable lead, and sensing the signals evoked by the tissue via a second implantable lead.

4. The method of claim 3, wherein the first implantable lead resides in one of a right ventricle and a left ventricle of the heart, and the second implantable lead resides in the other of the right ventricle and the left ventricle of the heart.

5. The method of claim 2, further comprising delivering the electrical stimulation to the tissue via an implantable lead, and sensing the signals evoked by the tissue via the implantable lead.

6. The method of claim 2, further comprising sensing the signals evoked by the tissue via an implantable lead, wherein evaluating sensing integrity comprises evaluating integrity of the implantable lead based on the analysis of the sensed signals.

7. The method of claim 1, wherein the stored data includes trend data for the sensed signals, and evaluating sensing integrity comprises evaluating sensing integrity based on the trend data.

8. The method of claim 7, wherein the trend data indicates at least one of a mean amplitude of the sensed signals over time or a mean time between delivery of the electrical stimulation and sensing of the evoked signals.

9. The method of claim 1, further comprising generating an indication of sensing integrity based on the evaluation.

10. The method of claim 1, wherein generating an indication of sensing integrity comprises generating the indication via at least one of the implantable medical device or an external programmer associated with the implantable medical device.

11. The method of claim 1, further comprising generating a notification in the event the evaluation indicates a sensing integrity condition.

12. The method of claim 1, further comprising sensing the signals via the implantable medical device, and evaluating the sensing integrity automatically within the implantable medical device.

13. The method of claim 1, further comprising sensing the signals via the implantable medical device, and evaluating the sensing integrity automatically within an external programmer associated with the implantable medical device.

14. The method of claim 1, wherein the implantable medical device includes multiple implantable leads, sensing signals comprises sensing signals via each of the implantable leads, and evaluating sensing integrity comprises evaluating lead integrity for each of the leads based on the analysis of the sensed signals.

15. The method of claim 14, wherein the leads comprise a right ventricular lead and a left ventricular lead, and the implantable medical device is configured to deliver cardiac resynchronization therapy (CRT) via the right and left ventricular leads.

16. The method of claim 1, further comprising:
sensing the evoked signals; and
storing data relating to the sensed evoked signals in memory of the implantable medical device.

17. The method of claim 1, further comprising sensing at least some of the evoked signals via a sensing vector including at least one high energy electrode as a sense electrode, wherein the high energy electrode is configured to deliver at least one of defibrillation or cardioversion stimulation.

18. An implantable medical device system comprising:
an implantable stimulation generator configured to deliver electrical stimulation to tissue;
an implantable sensing module configured to sense one or more signals evoked by the tissue in response to the electrical stimulation of the tissue by the implantable stimulation generator;
implantable memory configured to store data relating to the sensed signals; and
an evaluation unit configured to support evaluation of sensing integrity of the implantable sensing module based on analysis of the stored data.

19. The system of claim 18, wherein the implantable stimulation generator is configured to deliver cardiac pacing stimulation to cardiac tissue.

20. The system of claim 19, further comprising first and second implantable leads, wherein the stimulation generator is coupled to deliver the electrical stimulation via the first lead, and the sensing module is coupled to receive the sensed signals via the second implantable lead.

21. The system of claim 20, wherein the first implantable lead is one of a right or left ventricular lead and the second implantable lead is the other of the right or left ventricular lead.

22. The system of claim 19, further comprising an implantable lead, wherein the stimulation generator is coupled to deliver the electrical stimulation via the lead, and the sensing module is coupled to receive the sensed signals via the lead.

23. The system of claim 19, further comprising an implantable lead, wherein the sensing module is coupled to receive the sensed signals via the lead, and wherein the evaluation unit is configured to support evaluation of integrity of the lead based on the analysis of the stored data.

24. The system of claim 18, wherein the stored data includes trend data for the sensed signals, and the evaluation unit is configured to support evaluation of sensing integrity based on analysis of the trend data.

25. The system of claim 24, wherein the trend data indicates at least one of a mean amplitude of the sensed signals over time or a mean time between delivery of the electrical stimulation and sensing of the evoked signals.

26. The system of claim 18, wherein the evaluation unit is configured to generate an indication of sensing integrity based on the evaluation.

27. The system of claim 18, wherein the evaluation unit is implantable and is configured to automatically evaluate the sensing integrity, and wherein the stimulation generator, the sensing module and the evaluation unit form part of an implantable medical device.

28. The system of claim 18, further comprising an external programmer, wherein the evaluation unit forms part of the external programmer, and wherein the evaluation unit is configured to perform at least one of display of the stored data to a user or automatic evaluation of the sensing integrity.

29. The system of claim 28, wherein the evaluation unit is configured to perform automatic evaluation of the sensing integrity, and generate an indication of the sensing integrity to the user.

30. The system of claim 18, further comprising a notification module configured to generate a notification in the event the evaluation indicates a sensing integrity condition.

31. The system of claim 18, further comprising multiple implantable leads coupled to the sensing module to sense the signals via each of the implantable leads, wherein the memory is configured to store data relating to the sensed signals for each of the leads, and the evaluation unit is configured to support evaluation of lead integrity for each of the leads based on the analysis of the stored data.

32. The system of claim 31, wherein the leads comprise a right ventricular lead and a left ventricular lead, and the stimulation generator is configured to deliver cardiac resynchronization therapy (CRT) via the right and left ventricular leads.

33. The system of claim 31, wherein at least one of the leads comprises at least one high energy electrode as a sense electrode, the high energy electrode being configured to deliver at least one of defibrillation or cardioversion stimulation, and wherein the implantable sensing module is configured to sense the signals via a sensing vector comprising the high energy electrode.

34. An implantable medical device system comprising:
means for obtaining one or more sensed signals evoked by tissue in response to electrical stimulation of the tissue by an implantable medical device; and
means for evaluating sensing integrity of the implantable medical device based on analysis of the sensed signals.

35. The system of claim 34, wherein the tissue is cardiac tissue of a heart, and the electrical stimulation comprises cardiac pacing stimulation.

36. The system of claim 35, further comprising means for delivering the electrical stimulation to the tissue via an implantable lead, and means for sensing the signals evoked by the tissue via a second implantable lead, wherein the first implantable lead resides in one of a right ventricle and a left ventricle of the heart, and the second implantable lead resides in the other of the right ventricle and the left ventricle of the heart.

37. The system of claim 35, further comprising means for delivering the electrical stimulation to the tissue via an implantable lead, and means for sensing the signals evoked by the tissue via the implantable lead.

38. The system of claim 34, further comprising means for sensing the signals evoked by the tissue via an implantable lead, wherein the means for evaluating sensing integrity comprises means for automatically evaluating integrity of the implantable lead based on the analysis of the sensed signals.

39. The system of claim 34, wherein the implantable medical device includes multiple implantable leads, sensing signals comprises sensing signals via each of the implantable leads, storing data comprises storing data relating to the sensed signals for each of the leads, and evaluating sensing integrity comprises evaluating lead integrity for each of the leads based on the analysis of the stored data, and wherein the leads comprise a right ventricular lead and a left ventricular lead, and the implantable medical device is configured to deliver cardiac resynchronization therapy (CRT) via the right and left ventricular leads.

40. The system of claim 34, further comprising:
means for sensing the evoked signals; and
means for storing data relating to the sensed evoked signals in memory of the implantable medical device.

41. The system of claim 34, further comprising means for sensing at least some of the evoked signals via a sensing vector including at least one high energy electrode as a sense electrode, wherein the high energy electrode is configured to deliver at least one of defibrillation or cardioversion stimulation.

42. A computer-readable storage medium comprising instruction for causing a programmable processor to:
obtain one or more sensed signals evoked by tissue in response to electrical stimulation of the tissue by an implantable medical device; and
evaluate sensing integrity of the implantable medical device based on analysis of the sensed signals.
